(19) United States
(12) Patent Application Publication
(10) Pub. No.: US 2023/0144668 A1
(43) Pub. Date: May 11, 2023

(54) DIGITAL ANTIMICROBIAL STEWARDSHIP SYSTEM

(71) Applicant: Roche Molecular Systems, Inc., Pleasanton, CA (US)

(72) Inventors: Nishit S. Doshi, Santa Clara, CA (US); Jean M. Nelson, Foster City, CA (US); Brian P. Lee, Castro Valley, CA (US); Subita Sudershana, Oakland, CA (US)

(73) Assignee: Roche Molecular Systems, Inc., Pleasanton, CA (US)

(21) Appl. No.: 18/150,237

(22) Filed: Jan. 5, 2023

Related U.S. Application Data
(63) Continuation of application No. PCT/US21/41343, filed on Jul. 12, 2021.
(60) Provisional application No. 62/705,728, filed on Jul. 13, 2020.
(60) Provisional application No. 63/149,953, filed on Feb. 16, 2021.

(51) Int. Cl. G16H 20/10; G16H 70/20; G16H 50/70

(52) U.S. Cl. CPC G16H 20/10; G16H 70/20; G16H 50/70

(57) ABSTRACT

Digital antimicrobial stewardship program (ASP) systems and methods generate recommendations for a healthcare setting for treating a plurality of patients with a plurality of antibiotics, the recommendations based on medical data of the patients, antiobiogram information that indicate resistance of pathogens to the plurality of antibiotics within the healthcare setting, and a plurality of guidelines related to treatment of certain diseases using the plurality of antibiotics. The recommendation may include prescribing a dosage of an antibiotic or ordering a diagnostic test.

### CROSS REFERENCE TO RELATED APPLICATIONS

This patent application is a continuation of International Patent Application No. PCT/US2021/041343, filed Jul. 2, 2021, entitled “SYSTEMS AND METHODS FOR ANTIMICROBIAL STEWARDSHIP,” which claims the benefit of U. S. Provisional Patent Application No. 62/705,728, filed Jul. 13, 2020, entitled “SYSTEMS AND METHODS FOR ANTIMICROBIAL STEWARDSHIP,” and U. S. Provisional Patent Application No. 63/149,953, filed Feb. 16, 2021, entitled “SYSTEMS AND METHODS FOR ANTIMICROBIAL STEWARDSHIP,” which are assigned to the assignees hereof and are incorporated herein by reference in their entirety for all purposes.

### FIELD

Embodiments of this invention relate generally to antimicrobial stewardship, and in particular to antimicrobial stewardship in a health care setting.

### BACKGROUND

Antimicrobial resistance is expected to cause more deaths, estimated at about 10 million worldwide, than cancer by 2050. One problem is that more than 50% of antibiotics prescribed are unnecessary or inappropriate, which in turn causes more resistance to antibiotics. As a result, there is a significant rise in regulations and policies to implement antimicrobial stewardship programs (ASPs) in hospitals and health care settings around the world, to preserve the effectiveness of antibiotics, given the global health threat of antimicrobial resistance. An ASP is typically a coordinated program to promote the appropriate use of antibiotics to improve patient outcome and to reduce microbial resistance. The ASP typically involves collaboration between a treating team that prescribes and administers antibiotics to a patient as part of a therapy, and an ASP team that reviews the prescription/administration of the antibiotics and other patient information to recommend interventions to change the antibiotics therapy. The treating team can then implement the changes to the antibiotics therapy to optimize the therapy.

Although an ASP can play an important role in reducing antimicrobial resistance while optimizing the therapies for the patients, various sources of inefficiency may exist which can degrade the effectiveness of the ASP. For example, in order to make a clinical decision about which antibiotics to prescribe to a patient, or to intervene the prescription, a clinician may need to obtain different types of data from multiple databases, and select the data that is the most relevant for the clinical decision. But the sourcing of the data from the multiple databases, as well as the selection/identification of relevant data to make the clinical decision, is laborious, slow, and potentially error-prone. Moreover, typically the ASP team and the treating team collaborate in pre-scheduled meetings to determine whether to change an antibiotic therapy for a patient, but such arrangements can introduce a considerable delay between when the antibiotics therapy starts and when the antibiotics therapy is changed. All these can substantially degrade the effectiveness of the ASP in reducing antimicrobial resistance and improving the quality of care provided to the patients.

### BRIEF SUMMARY

Examples of the present disclosure provide a digital ASP system that can address at least some issues to improve the execution of an ASP. Specifically, the digital ASP system can include one or more ASP team sub-systems and one or more treating team sub-systems. The ASP team sub-system can provide the ASP team access to relevant information for a clinical decision to intervene in the prescription of antibiotics and/or a diagnostic test order by the treating team, and transmit the intervention recommendation to the treating team sub-system via real-time communication. The ASP team sub-system can also generate a report to record various statistics, to help administrators to evaluate the execution of the ASP. The ASP team sub-system can also send the report to an external agency, such as National Healthcare Safety Network (NHSN) of Centers for Disease Control and Prevention (CDC).

Moreover, the treating team sub-system can provide the treating team access to relevant information for a clinical decision to prescribe antibiotics to a patient. The treating team sub-system can also receive the intervention recommendation from the ASP sub-system, transmit a response to the intervention recommendation back to the ASP sub-system, and transmit prescription orders to other treating team sub-systems via real-time or asynchronous communication. The ASP team sub-system can be accessible by the ASP team via a first interface (hereinafter, an ASP team interface). In some examples, the ASP team interface can be a desktop interface to be provided on a display screen of a computer. In some examples, the ASP team interface can be a mobile interface to be provided on a mobile device of a member of the ASP team. Moreover, the treating team sub-system is accessible by the treating team via a second interface (hereinafter, a treating team interface), which can be provided on a mobile device of a member of the treating team.

These and other embodiments of the invention are described in detail below. For example, other embodiments are directed to systems, devices, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

The detailed description is set forth with reference to the accompanying figures.

FIG. 1, FIG. 2A, FIG. 2B, and FIG. 2C illustrate a conventional antimicrobial stewardship program (ASP) and the information involved in the ASP.

FIG. 3A-FIG. 3H illustrate example components of a digital ASP system, according to certain aspects of the present disclosure.

FIG. 4A-FIG. 4E illustrate examples of an ASP team interface provided by the digital ASP system, according to certain aspects of the present disclosure.

FIG. 5A-FIG. 5D illustrate examples of a treating team interface provided by the digital ASP system, according to certain aspects of the present disclosure.

FIG. 6A and FIG. 6B illustrate examples of methods to support an ASP, according to certain aspects of the present disclosure.

FIG. 7 illustrates an example computer system that may be utilized to implement techniques disclosed herein.

### DETAILED DESCRIPTION

An antimicrobial stewardship program (ASP) is typically a coordinated program to promote the appropriate use of antibiotics to improve patient outcome and to reduce antimicrobial resistance. The ASP typically involves collaboration between a treating team that prescribes and administers antibiotics to a patient as part of a therapy, and an ASP team that reviews the prescription/administration of the antibiotics and other patient information to recommend interventions to change the antibiotics therapy. The treating team can then implement the changes to optimize the therapy.

The effectiveness of an ASP in reducing antimicrobial resistance and optimizing the therapies for the patients, however, can be degraded by various sources of inefficiency. Specifically, a clinician typically needs to procure different types of information, such as medical data of the patient (e.g., medical history, lab data, etc.), guidelines/regulations that govern the prescription, characteristics information of the antibiotics such as antibiogram information, etc., which are typically stored in multiple databases. Moreover, the clinician may also need to select the data that is the most relevant for the clinical decision. The sourcing of the data from the multiple databases, as well as the selection/identification of relevant data to make the clinical decision, is laborious, slow, and potentially error-prone. In addition, typically the ASP team makes a clinical decision about intervening the prescription of antibiotics by the treating team, and the treating team receives the intervention recommendations, only during pre-scheduled meetings. Therefore, after a patient starts an antibiotic therapy, the patient needs to wait until the next pre-scheduled meeting between the ASP team and the treating team before any changes can be made to the therapy. As a result, there can be substantial delay in implementing changes in the antibiotics therapy for a patient, and the intervening is typically reactive rather than proactive in nature. All these can degrade the effectiveness of the ASP in reducing antimicrobial resistance and improving the quality of care provided to the patients.

Examples of the present disclosure provide a digital system to improve the execution of an antimicrobial stewardship program (ASP). Specifically, the digital system can include one or more ASP team sub-systems and one or more treating team sub-systems. The ASP team sub-system can provide the ASP team access to relevant information for a clinical decision to intervene the prescription of antibiotics and/or a diagnostic test (e.g., a diagnostic test related to a bacterial infection such as staining and examination, culture, testing of pathogen's susceptibility/sensitivity to antibiotics, etc.) ordered by the treating team, and transmit an intervention recommendation based on the ASP team's clinical decision to the treating team sub-system via real-time or asynchronous communication. The ASP team sub-system can also generate a report to record various statistics, such as the prescription of antibiotics, interventions, etc., to help administrators to evaluate the execution of the ASP. The ASP team sub-system can also send the report to an external agency, such as National Healthcare Safety Network (NHSN) of Centers for Disease Control and Prevention (CDC).

Moreover, the treating team sub-system can provide the treating team access to relevant information for a clinical decision to prescribe antibiotics and/or to order diagnostic tests to a patient. The treating team sub-system can also transmit a prescription order based on the clinical decision to other treating team sub-systems. The treating team sub-system can also receive the intervention recommendation from the ASP sub-system, transmit a response to the intervention recommendation back to the ASP sub-system, and transmit prescription orders (for antibiotics, diagnostic tests, etc.) to other treating team sub-systems via real-time or asynchronous communication.

Specifically, the ASP team sub-system can be accessible via an ASP team interface. In some examples, the ASP team interface can be a desktop interface to be provided on a display screen of a computer. In some examples, the ASP team interface can also be a mobile interface to be provided on a mobile device. The ASP team interface can include an ASP team data access interface and an ASP team communication interface. The ASP team sub-system can receive various medical data of patients that are relevant or needed for an intervention recommendation from one or more databases over a network, and aggregate the data. The intervention recommendation can be to change an antibiotic prescription, change a diagnostic test related to bacterial infection, etc. The aggregated medical data of patients can include, for example, the medical history of the patients, their most recent diagnoses, medications including antibiotics, results of various measurements (e.g., body temperatures, blood pressures, etc.), and results of various laboratory tests (e.g., bacterial testing, culturing bacteria, antibiotic sensitivity testing, gram stain, etc.). The ASP team sub-system can also aggregate other data relevant for the intervention recommendation, such as antibiogram information, guidelines for prescription and administering of the antibiotics, etc.

The ASP team sub-system can receive a trigger to retrieve and aggregate patients data for ASP intervention determination. The trigger can be based on, for example, a command from the ASP team to access the medical data of certain patients, a timer that indicates a review of patients' antibiotics usage/prescription by the ASP team is due, new antibiotics prescriptions have been entered for certain patients and the prescriptions have not been reviewed, etc.

The ASP team sub-system can then provide the aggregated data to the ASP team data access interface for displaying, or otherwise make the aggregated data accessible via the ASP team data access interface, so that an ASP team member can access all the data needed to make an intervention recommendation from the ASP team data access interface instead of accessing the data from different sources and/or in different interfaces. In some examples, the ASP team data access interface can also select a subset of the aggregated data, and display the selected data in the ASP team data access interface. In some examples, the ASP team sub-system can automatically perform a filtering operation based on a degree of relevancy of the data to a particular patient. In some examples, the filtering operation can also be performed based on inputs from the user.

In addition, to facilitate efficient review and intervention of the patients' therapies/tests, the ASP team sub-system can automatically select a subset of patients based on the antibiotic profile, laboratory test results of the patients, medical history of the patients, intervention tracking status, etc. The selection can be based on a scoring system. Specifically, the ASP team sub-system can determine a triage score for each patient, rank the patients based on their triage scores, and display a ranked list of patients in the ASP team interface. The triage score can indicate the urgency for reviewing the patient's treatment/test. The ASP team can then refer to the ranking to select a patient for review. The triage score can be a weighted average of various factors (e.g., availability of the most recent lab test result, lab/drug mismatch, restricted drug prescription, etc.), to indicate the urgency for reviewing the patient's antibiotics therapy. The weights can be determined by the ASP team and/or automatically by the ASP team sub-system based on, for example, prior interventions. The ASP team interface can receive a selection from the ASP team of a particular patient in the ranked list, and display the aforementioned filtered data of the selected patient in the ASP team data access interface.

In addition to displaying the relevant data for an intervention recommendation, the ASP team sub-system can also generate, as part of a clinical decision support (CDS), an intervention recommendation to facilitate the decision. The ASP team sub-system can generate the recommendation based on, for example, the medical data of the selected patient, antibiogram information, guidelines, etc., in response to a trigger. In some examples, the trigger may be the same trigger that causes the ASP team sub-system to retrieve and aggregate patients data for ASP intervention determination. In some examples, the trigger can be a different trigger based on, for example, an indication from the databases that new medical data of a patient being reviewed (e.g., lab test results, or other new medical data that have not been processed by the sub-system) are available. The indication can be in the form of a network message transmitted by the databases in response to a query transmitted by the ASP team sub-system.

The ASP team sub-system can display the recommendation in the ASP team data access interface concurrently with the medical data, to enable the user to have access to the basis of the recommendation. The ASP team sub-system can also generate a notification of the intervention recommendation, which can be generated automatically based on the recommendation, or based on an input from the ASP team, and transmit the notification to the treating team sub-system via real-time communication, such as text-messaging, voice call, etc. The notification can also be a snoozing notification to be responded to by the treating team member at a later time as part of asynchronous communication. The ASP team sub-system can also track (automatically and/or based on inputs from the ASP team) the status of an intervention recommendation (e.g., whether a therapy change has been implemented, a diagnostic test has been ordered, etc.). In some examples, the ASP team communication interface can be in the form of a text-messaging interface, in which the notification, as well as a response to the notification from the treating team sub-system, can be displayed in the form of text messages. The ASP team sub-system can display the ASP team data access interface and the ASP team communication interface concurrently, which allows the users to communicate via text messages or voice while having access to the medical data, to facilitate the collaboration experience. All the information needed to act on the notification is also provided in the treating team interface.

In addition, the ASP team sub-system can include additional functionalities to support the ASP. For example, the ASP team sub-system can generate a report to record various statistics, to help administrators to evaluate the execution of the ASP. Such a report may include, for example, a report on days of therapy, or an intervention acceptance report. The ASP team sub-system can also send the report to an external agency, such as National Healthcare Safety Network (NHSN) of Centers for Disease Control and Prevention (CDC). As another example, the ASP team sub-system can support various administrative functions, such as configuring the generation of notifications, setting access rights to the notification (e.g., which member of the ASP team can generate/receive notification, which member of the treating team can receive a notification from the ASP team sub-system via the treating team sub-system, etc.), editing/abstracting the medical data (e.g., editing of the guidelines, antibiogram, converting the data into proprietary formats, etc.).

Moreover, the treating team sub-system is accessible by the treating team via a treating team interface, which can be provided on a mobile device of a member of the treating team. The treating team interface can include a treating team data access interface and a treating team communication interface. The treating team sub-system can also retrieve various medical data that are relevant (or needed) for a clinical decision for a particular patient from one or more databases over a network, and aggregate the data. The treating team sub-system can retrieve and aggregate the medical data based on receiving a trigger, such as a selection from the treating team to review the medical history of the patient.

The clinical decision can include, for example, a first dose or an empiric therapy, a diagnostic test related to bacterial infection (e.g., a diagnostic test related to a bacterial infection such as staining and examination, culture, identification of pathogen, testing of pathogen's susceptibility/sensitivity to antibiotics), etc. The medical data can include, for example, the vitals and laboratory test results of the particular patient, antibiogram information, guidelines, etc. The treating team sub-system can then provide the aggregated data to the treating team data access interface for display, or otherwise provide access to the aggregated data via the treating team data access interface. This allows a treating team member to access all the data needed to make a clinical decision (e.g., about a first dose, an empiric therapy, ordering a diagnostic test related to bacterial infection) from the treating team data access interface instead of accessing the data from different sources and/or in different interfaces.

In addition, the treating team sub-system can also automatically perform a filtering operation on some of the data, such as antibiogram information, based on a degree of relevancy of the data to the patient. For example, the treating team interface can provide antibiogram information based on a location where the patients are to be administered antibiotics (e.g., ICU). As another example, the treating team sub-system can automatically select a subset of patients based on the antibiotic profile, laboratory test results of the patients, medical history of the patients, intervention tracking status, etc., and provide access to the medical data of the subset of patients via the treating team data access interface. In some examples, the filtering operation can also be performed based on inputs from the user.

In addition to displaying the relevant data for a clinical decision about a first dose or an empiric therapy, the treating team sub-system can also generate, as part of CDS, a recommendation for the first dose or the empiric therapy. The treating team sub-system can generate the recommendation based on various types of medical data, such as a medical history of the patient including allergies to drugs, a suspected diagnosis of the patient, suspected pathogens causing a disease of the patient, a risk of drug resistance of the patient, laboratory test results of the patient, antibiogram information, guidelines, formulary restrictions and inventory status etc. The recommendation can be generated based on, for example, receiving a command/request from a treating team member, receiving an indication (e.g., a network message) that new medical data (e.g., laboratory test results) of the patient is available, etc. The treating team sub-system can display the recommendation in the treating team data access interface, to enable the user to have access to the basis of the recommendation. The treating team can then make a clinical decision (e.g., a prescription order) based on accepting or rejecting the recommendation.

In addition, the treating team sub-system can also generate/receive a notification of a clinical decision. The notification (which can include snoozing notification, text messages, etc.) can be generated based on an input from a clinician of the treating team, and transmit the notification to other treating team sub-systems operated by other members of the treating team (e.g., pharmacists) via real-time communication, such as text-messaging, voice call, etc. In addition, the treating team sub-system can receive a notification of intervention from the ASP sub-system, as described above. In some examples, the notifications can cause the mobile device to generate a sensory output (e.g., a vibration, a tone, etc.) to alert the user about the notifications. In addition, the treating team sub-system can activate the treating team interface upon receiving a selection of a notification from the user. In some examples, the treating team communication interface can be in the form of a text-messaging interface, in which the notification, as well as a response to the notification from other treating team sub-systems, can be displayed in the form of text messages. The treating team sub-system can display the treating team data access interface and the treating team communication interface concurrently, which allows the users to communicate via text messages or voice while having access to the medical data, to facilitate the collaboration experience. The treating team interface also allows snoozing notifications so the treating team can act on the notifications at a later time.

With a digital ASP system according to examples of the present disclosure, both the ASP team and the treating team can have access to all the relevant information, as well as system-generated recommendations, for a clinical decision (intervention recommendation, prescription of first dose/empiric therapy, etc.) from a single data access interface. Compared with a case where the clinicians need to source different data from different systems, or need to access the different data via different interfaces, the digital ASP system can substantially improve the clinicians' access to the data, which not only speeds up the clinical decisions but also improves the quality of the decisions. Moreover, the triage ranking provided by the digital ASP system can ensure that patients whose therapies/diagnostic tests need to be reviewed and/or changed can get the intervention in a timely manner. Further, by providing real-time communication capability between the ASP team and the treating team, the intervention recommendation by the ASP team can be communicated to the treating team in real-time and at anywhere (e.g., on the bedside of the patient, at the pharmacy department, etc.) as soon as when the intervention recommendation is made. Compared with a case where the ASP team and the treating team only collaborate during pre-scheduled meetings, such arrangements can further speed up the intervention of a patient's therapy and diagnostic tests. All these can improve the quality of care provided to the patients, as well as the effectiveness of ASP in reducing antimicrobial resistance.

### I. Examples of an Antimicrobial Stewardship Program (ASP)

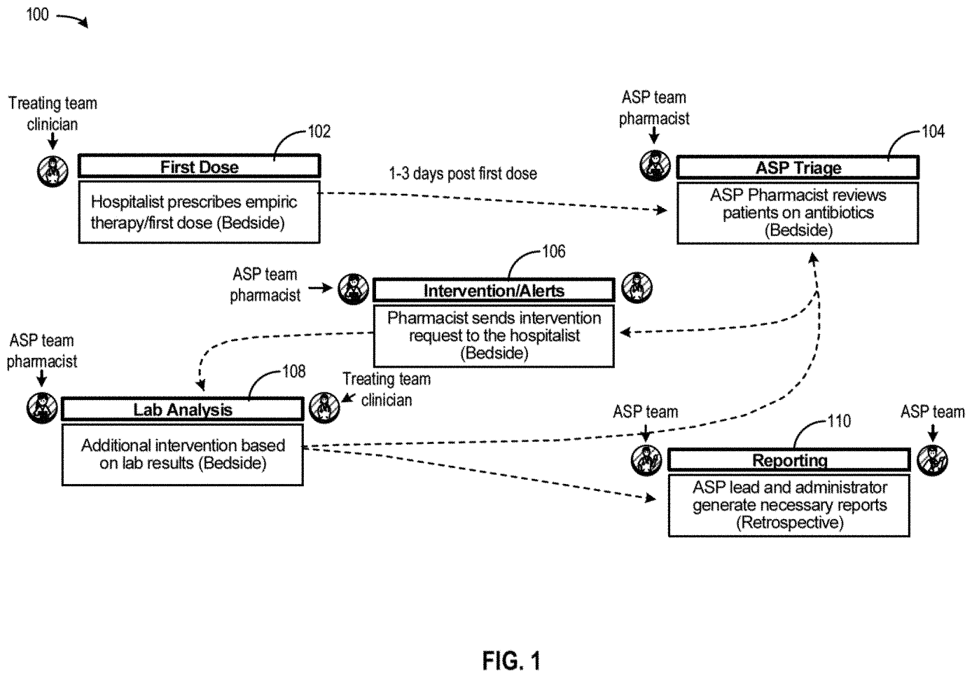

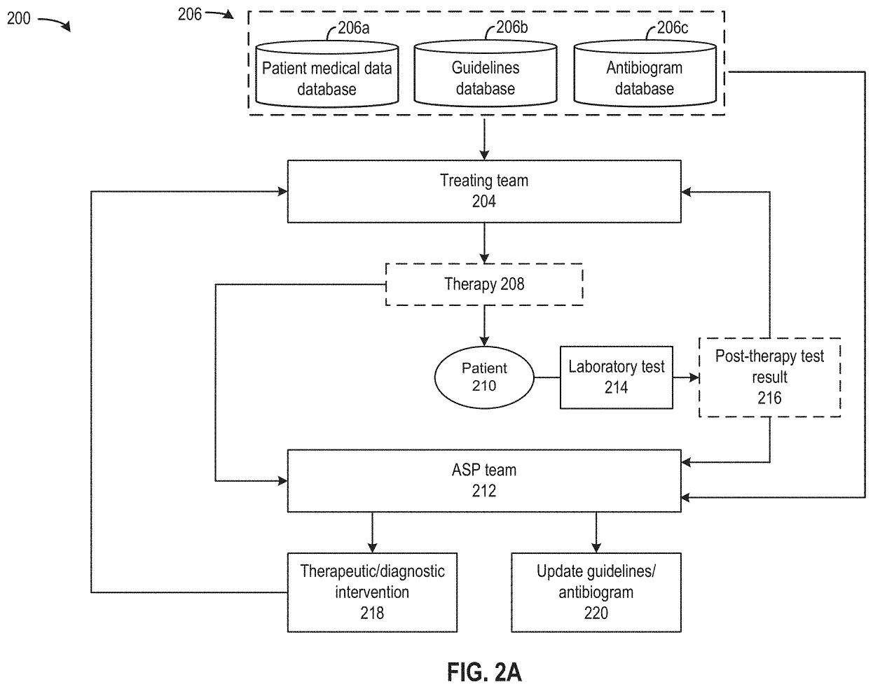

FIG. 1, FIG. 2A, FIG. 2B, and FIG. 2C illustrate examples of an antimicrobial stewardship program (ASP). An ASP is typically a coordinated program to promote the appropriate use of antibiotics to improve patient outcome and to reduce microbial resistance. The ASP typically involves collaboration between a treating team that prescribes and administers antibiotics to a patient as part of a therapy, and an ASP team that reviews the prescription/administration of the antibiotics to recommend interventions to change the antibiotics therapy.

A. Example Interactions Between an ASP Team and a Treating Team

FIG. 1 illustrates an example ASP program 100. As shown in FIG. 1, in step 102, the treating team (hospitalist in FIG. 1) prescribes an empiric therapy or a first dose of antibiotics to a patient. The first dose typically refers to initial antibiotics prescribed to the patient with suspected infection, whereas empiric therapy typically refers to antibiotics given to the patient prior to determination of the causative pathogen. About 1-3 days after the first dose is administered, an ASP team member, such as an ASP team pharmacist and/or clinician, can review the patients' antibiotics therapies, in step 104. The review can be performed at a pre-scheduled meeting between the ASP team and the treating team. As part of the review process, the patients can be triaged based on priority levels. Patients deemed to be high priority can have their antibiotics therapies be reviewed by the ASP team pharmacist to identify opportunities to optimize the antibiotics therapies. If the ASP team identifies an opportunity to change the antibiotics, the ASP team can send an intervention request to the treating team, in step 106, who can then change the antibiotics therapy prescribed to the patient. The intervention request can also seek to change the diagnostic/laboratory test administered to the patient. After the patient receives the updated antibiotics therapy, new laboratory results may be received for the patient, in step 108. Additional review and intervention can be generated in step 104 by the ASP team based on the laboratory tests results. In addition, in step 110 the ASP team can generate retrospective reports of antibiotics usage at the hospital, including the prescription of the antibiotics to the patients and subsequent intervention.

B. Example Flow Diagram of an ASP Program

FIG. 2A, FIG. 2B, and FIG. 2C illustrate additional details of an example ASP program 200. FIG. 2A illustrates a flow diagram of ASP program 200. As shown in FIG. 2A, a treating team 204 can source various data from one or more databases 206 to determine a therapy 208 for a patient 210. Databases 206 can include, for example, a medical data database 206a, a guidelines database 206b, and an antibiogram database 206c. Therapy 208 can be a first dose/empiric therapy, or an updated therapy based on an intervention recommendation from ASP team 212, and can correspond to step 102 of FIG. 1.

Medical data database 206a may store data including the medical data of a patient. The medical data may include, for example, a recent diagnosis of which pathogen(s) cause an infection at patient 210, medical history, laboratory tests results, body measurements (e.g., blood pressure, body temperature, etc.) results, etc. Guidelines database 206b can store guidelines for prescribing different antibiotics for different diseases, at different settings (e.g., whether the patient is admitted to a hospital or not), for different medical conditions (e.g., whether other comorbidities are present, whether the patient is immunocompromised), etc., to determine whether the prescription of the antibiotics is appropriate and consistent with the guidelines. Guidelines database 206b may also store links to national guidelines such as, for example, Center for Disease Control and Prevention (CDC) core elements of antimicrobial stewardship.

An example of guidelines 207 stored in guidelines database 206b is illustrated in FIG. 2B. As shown in FIG. 2B, guidelines 207 can list the antibiotics to be used to treat a particular disease, such as community-acquired pneumonia (CAP). Guidelines 207 include a section 207-a and a section 207-b for patients not admitted to the hospital and for different levels of comorbidities, as well as a section 207-c and a section 207-d for patients admitted to the hospital and for different levels of comorbidities. Depending on whether a patient is admitted to a hospital, and the kind of comorbidity and its severity present in the patient, a particular antibiotic can be prescribed according to one of sections 207-a, 207-b, 207-c, or 207-d.

In addition, antibiogram information stored in antibiogram database 206c can document the susceptibility/resistance of various pathogens to a variety of antibiotics at different settings (e.g., inpatient, outpatient, intensive care unit (ICU), long-term care facilities, etc.). An example of antibiogram table 209 stored in antibiogram database 206c is illustrated in FIG. 2C. As shown in FIG. 2C, antibiogram table 209 lists the percentage susceptibility of a pathogen to list of antibiotics in an ICU. For example, referring to section 209-a, the pathogen pseudomonas aeruginosa is 78% susceptible to cefepime and is 67% susceptible to aztreonam, which can indicate that pseudomonas aeruginosa is more resistant to aztreonam than to cefepime. Moreover, referring to section 209-b, the pathogen enterobacter cloacae is 100% susceptible to imipenem but is 0% susceptible to ampicillin, which can indicate that enterobacter cloacae is completely resistant to ampicillin but can be effectively eliminated by imipenem.

After patient 210 receives therapy 208, the patient can undergo a laboratory test 214 to obtain a post-therapy test result 216. Laboratory test 214 may include various tests, such as bacterial testing, culturing bacteria, antibiotic sensitivity testing, gram stain, etc., to determine the pathogens present in patient 210 and their resistance to antibiotics. Post-therapy test result 216 can be used to gauge whether antibiotics therapy 208 is effective in reducing the level of pathogen, whether the antibiotics therapy 208 increases the resistance of the pathogen, etc.

Post-therapy test result 216 can then be provided to both treating team 204 and ASP team 212. Treating team 204 can analyze post-therapy test result 216 and determine whether to change antibiotics therapy 208 prescribed to patient 210. Treating team 204 may determine to change antibiotics therapy 208 if, for example, antibiotics therapy 208 is not effective in reducing the level of pathogen due to microbial resistance. Treating team 204 can perform the analysis, and determine to change the therapy, in a pre-scheduled visit to patient 210.

Moreover, ASP team 212 can also conduct a review meeting with treating team 204 to review, for example, antibiotics therapy 208 by treating team 204, various diagnostic/laboratory tests ordered by treating team 204 for patient 210, etc. The review can be based on various types of information. The review can be based on post-therapy test result 216 as well as data in databases 206. For example, a determination can be made about whether antibiotics therapy 208 is appropriate according to guidelines retrieved from guidelines database 206b, whether antibiotics therapy 208 is effective in eliminating the targeted pathogen according to antibiogram information retrieved from antibiogram database 206c, etc., and even if it does, whether antibiotics therapy 208 can be further optimized to reduce microbial resistance. As another example, a determination can be made about whether the diagnostic/laboratory tests are effective in measuring the level of pathogens present in patient 210, the level of microbial resistance of the pathogens present in patient 210, etc. The review can correspond to step 104 of FIG. 1 and can be performed at a pre-scheduled meeting between treating team 204 and ASP team 212. As a result of the review, ASP team 212 can generate a therapeutic/diagnostic intervention 218, which can be provided to treating team 204 during the meeting to alter therapy 208 and/or diagnostic/laboratory tests to be ordered by treating team 204 for patient 210. In addition, if the review indicates a new type of microbial resistance, ASP team 212 can also generate an update 220 to guidelines database 206b and/or antibiogram database 206c. Treating team 204 can then use the updated guidelines in guidelines database 206b and the updated antibiogram information in antibiogram database 206c to determine a subsequent antibiotics therapy for patient 210 or for other patients.

C. Example Factors Affecting the Effectiveness of an ASP Program

The effectiveness of ASP 200 in reducing antimicrobial resistance and optimizing the therapies for the patients, however, can be degraded by various sources of inefficiency. Specifically, as described above, both treating team 204 and ASP team 212 may need to procure data from databases 206. The data include different types of information such as medical data of a patient, guidelines, and antibiogram information and are typically stored in multiple databases. Moreover, typically only a small subset of the procured data is relevant for the clinical decision. The sourcing of the data from the multiple databases, as well as the selection/identification of relevant data to make the clinical decision, is laborious, slow, and potentially error-prone.

For example, referring to FIG. 2B, a clinician who tries to refer to guidelines for administering antibiotics for a particular disease needs to retrieve the guidelines for that disease (e.g., guidelines 207 for CAP, other guidelines for other diseases), and then refer to the relevant portion of the guidelines based on the medical condition of the patient (e.g., one of sections 207-a, 207-b, 207-c, or 207-d) to obtain the relevant guideline. As another example, referring to FIG. 2C, a clinician who tries to select an antibiotic to which a particular pathogen is most susceptible needs to retrieve the antibiogram for a particular setting where the patient is to receive the antibiotic (ICU, inpatient, or outpatient), and then select the section corresponding to the pathogen. The clinician may also need to cross-reference with other information, such as the inventory of antibiotics, the cost of antibiotics, etc., to make a decision about which antibiotics to prescribe. As a result, the clinician needs to parse through a huge volume of guidelines and antibiogram information, pick a small subset of the information that is most relevant for a particular patient at a particular setting, cross-reference with the various medical data of a patient, and then make a clinical decision about prescribing an antibiotic (or changing a prescription) for that patient. Coupled with the fact that the patient's medical data, the guidelines, and the antibiogram information are typically stored in different places, and that clinical decisions about antibiotics therapy need to be made for a large number of patients, it becomes challenging for a clinician to find the relevant information to make a proper clinical decision for each patient in an efficient manner. The effectiveness of ASP, as well as the quality of care provided to the patients, can become degraded as a result.

In addition, the conventional way of collaboration between the ASP team and the treating team can also degrade the effectiveness of ASP. Specifically, typically the ASP team makes a clinical decision about intervening the prescription of antibiotics by the treating team, and the treating team receives the intervention recommendations, only during pre-scheduled meetings. Similarly, the treating team typically reviews the patient's laboratory data and makes a decision to change the patient's antibiotics therapy during pre-scheduled visits to the patients. Therefore, after a patient starts an antibiotic therapy, the patient needs to wait until the next scheduled meeting between the ASP team and the treating team, or the next scheduled visit by the treating team, before any changes can be made to the therapy. As a result, there can be substantial delay in implementing changes in the antibiotics therapy for a patient, and the change is typically reactive (e.g., after seeing substantial degradation in the patient's vitals) rather than proactive in nature. All these can further degrade the effectiveness of ASP and the quality of care provided to the patients.

### II. Digital System to Support ASP

A. System Overview

Examples of the present disclosure provide a digital ASP system that can facilitate ASP management as well as prescription of medical treatments and/or diagnoses. The digital system can include one or more ASP team sub-systems and one or more treating team sub-systems. The ASP team sub-system can provide the ASP team access to curated/filtered information of patients to determine whether to intervene the prescription of antibiotics and/or diagnostic tests for the patients, and allow the ASP team to transmit intervene decisions to the treatment team. The ASP team sub-system can also provide the ASP team access to a list of high-priority patients and their information. The ASP team sub-system can also analyze the information and provide recommendation to the ASP team for the intervene decision. Moreover, the treating team sub-system can also provide the treating team access to curated/filtered information of patients to prescribe antibiotics/diagnostic tests. The treating team sub-system can also receive and display intervene decisions. The treating team sub-system can also generate recommendations for the prescriptions.

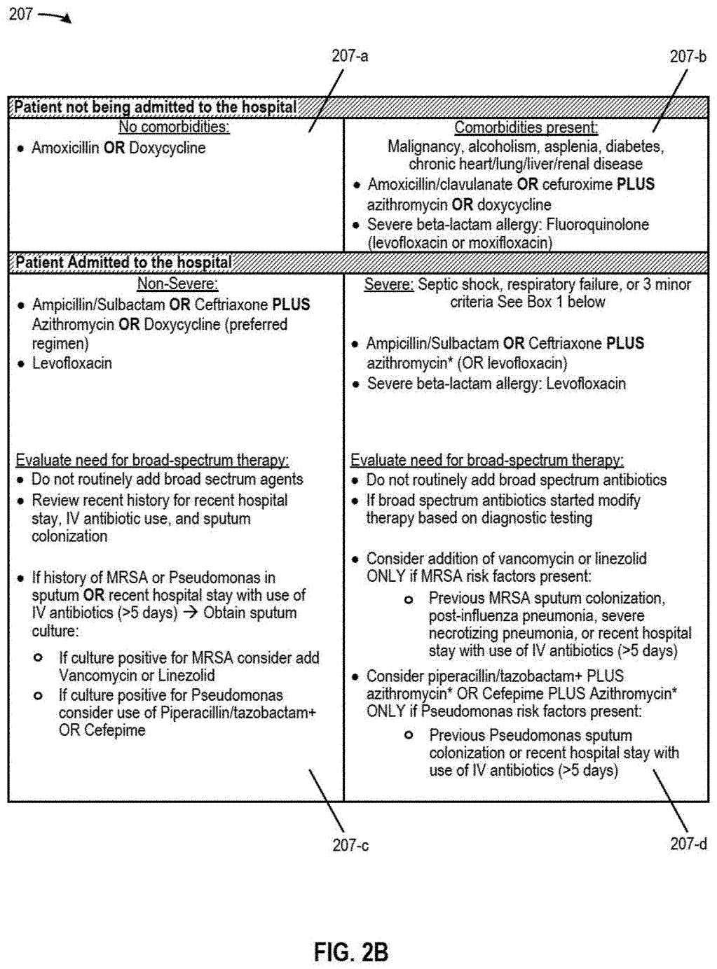

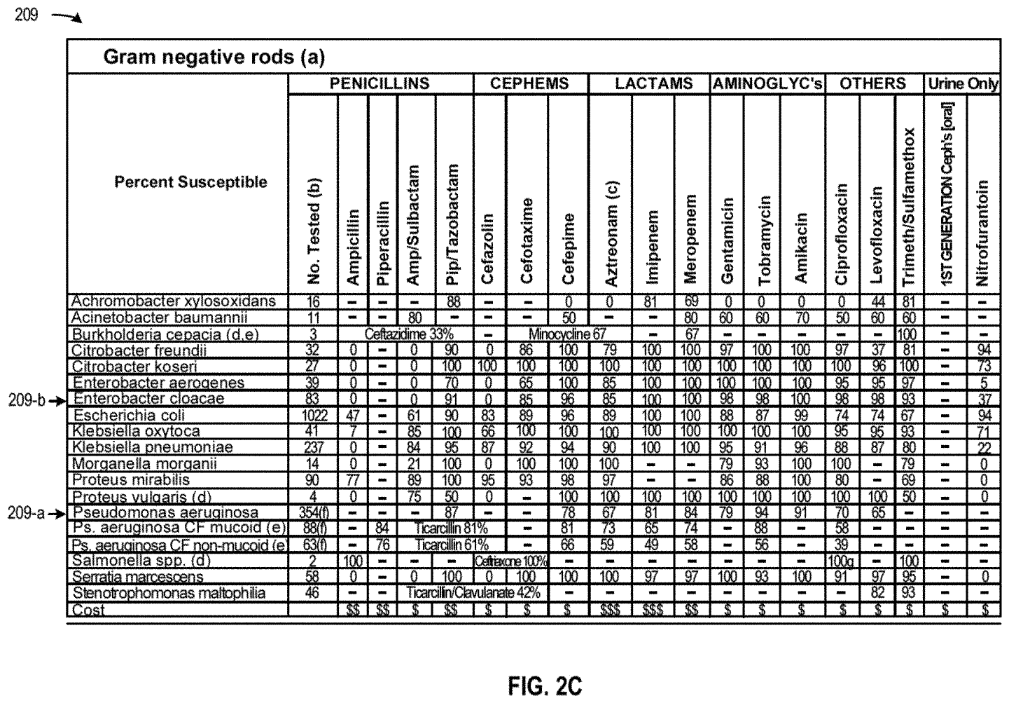

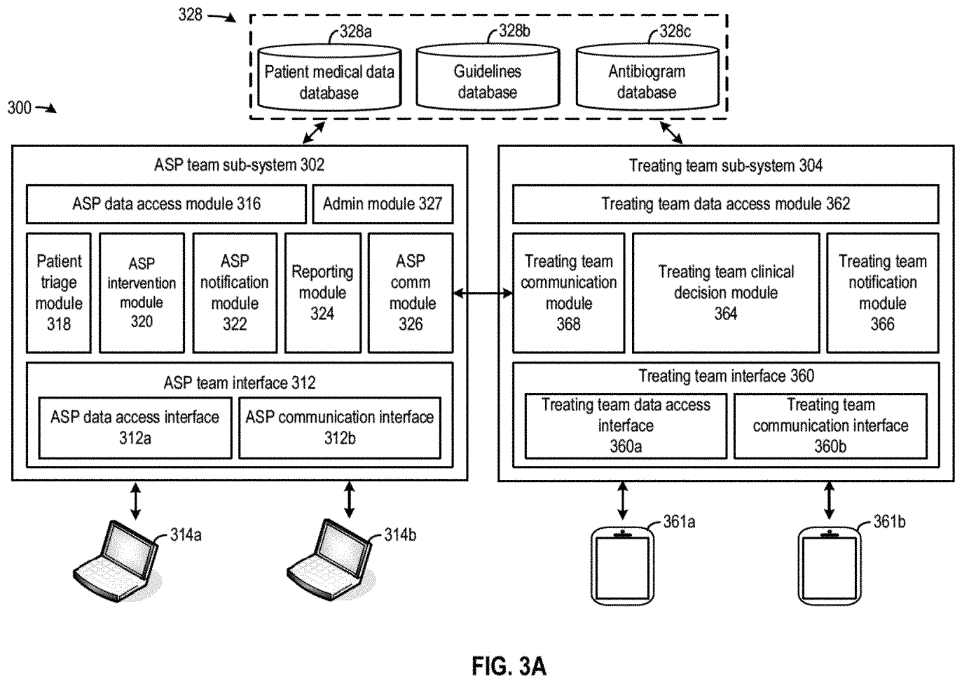

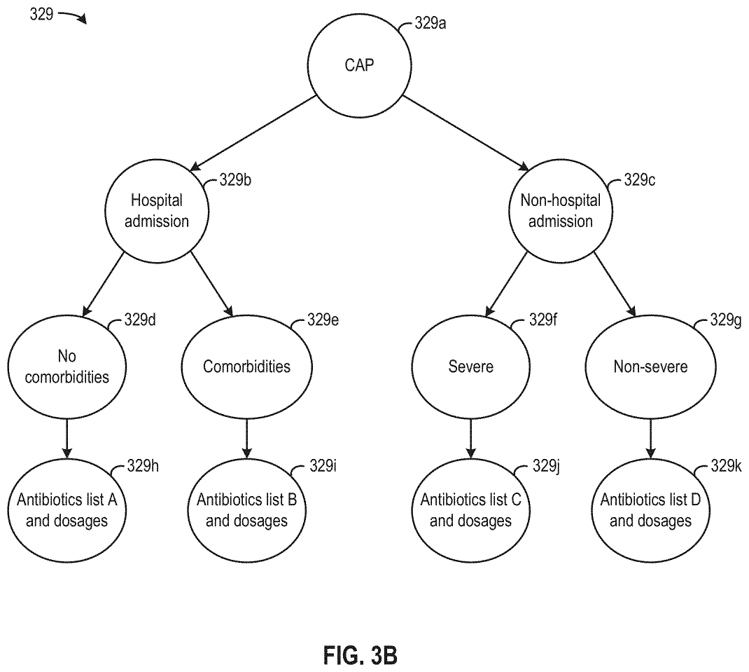

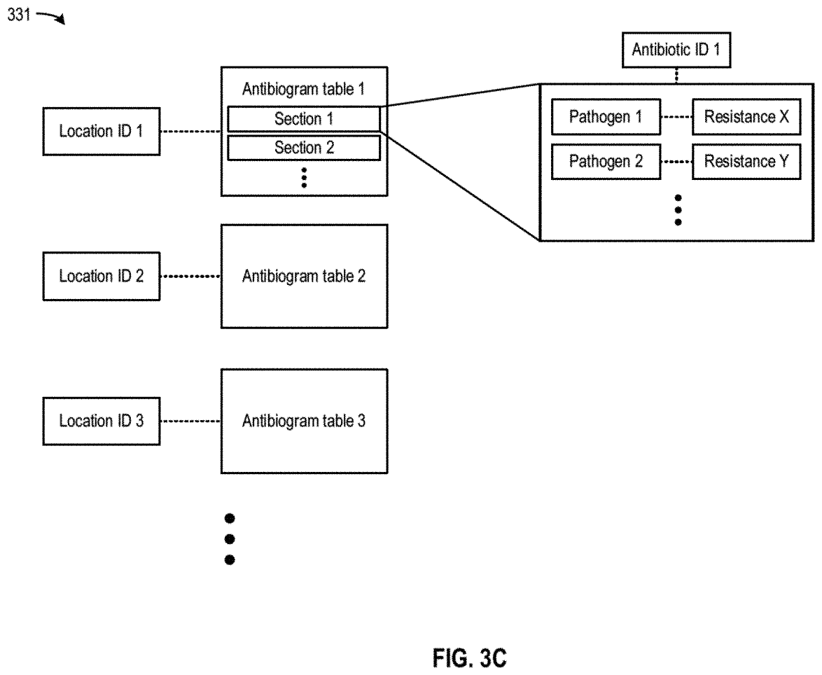

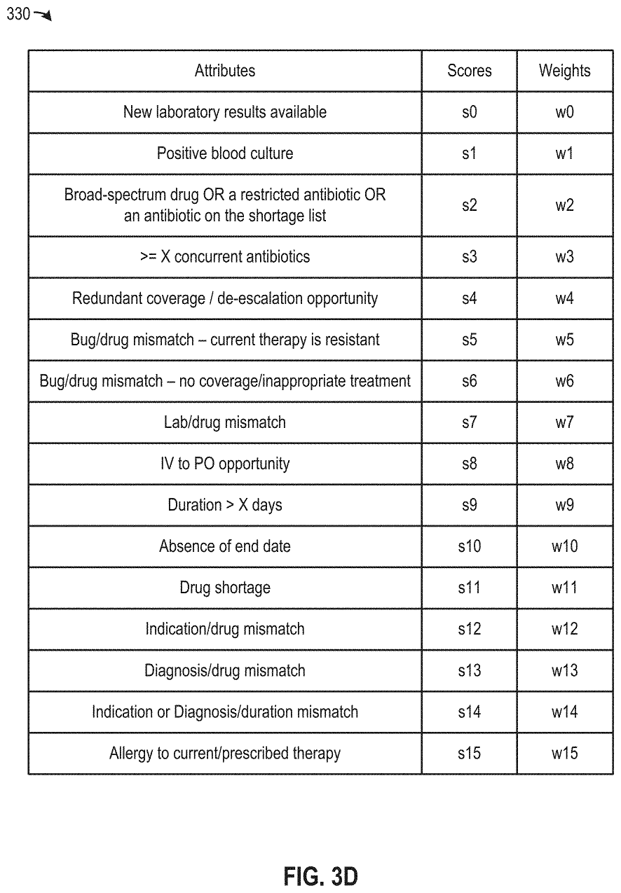

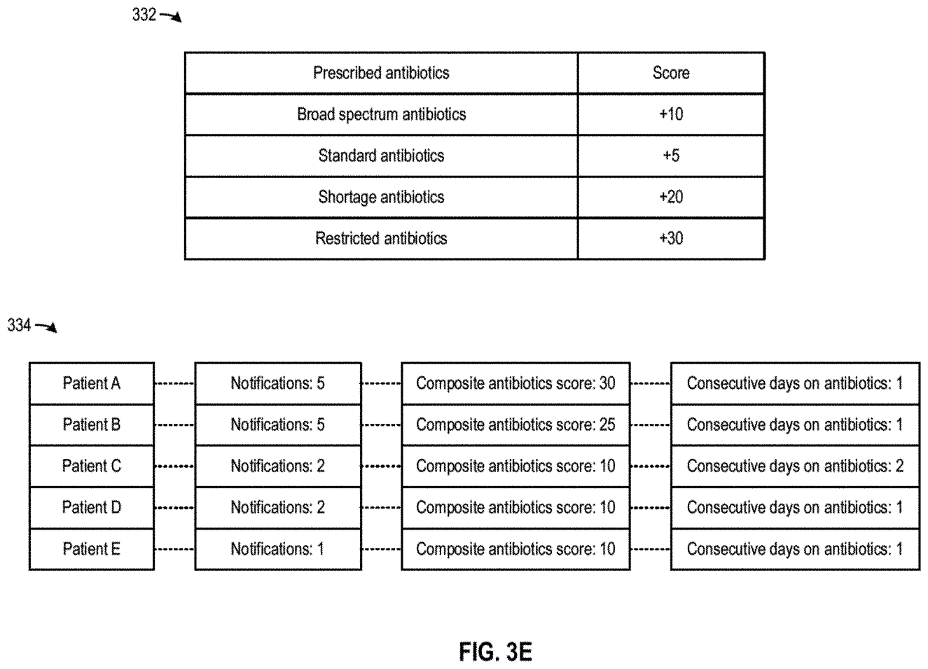

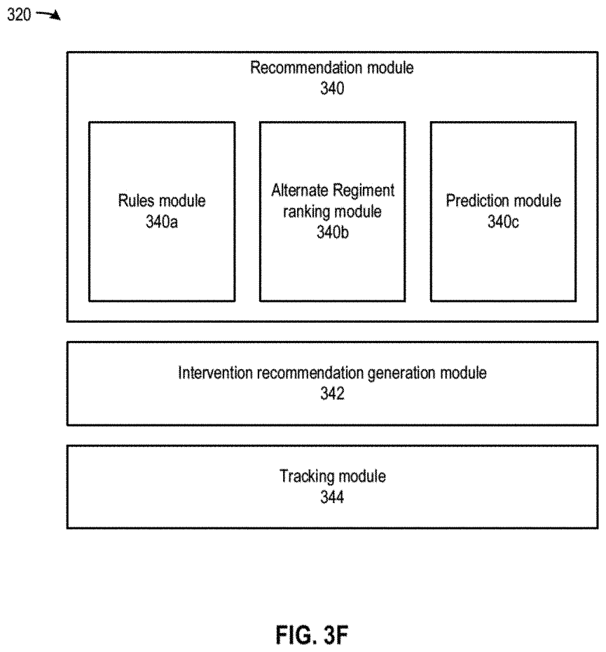

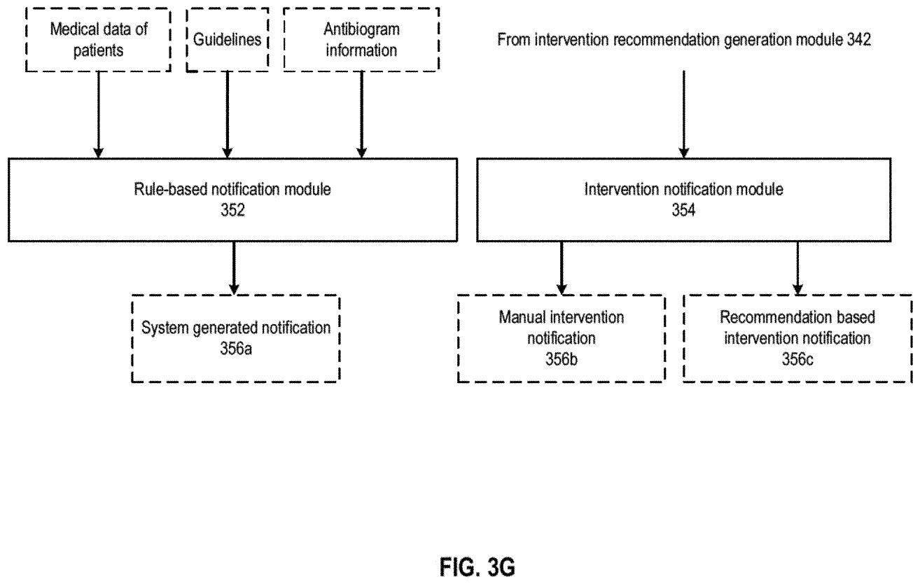

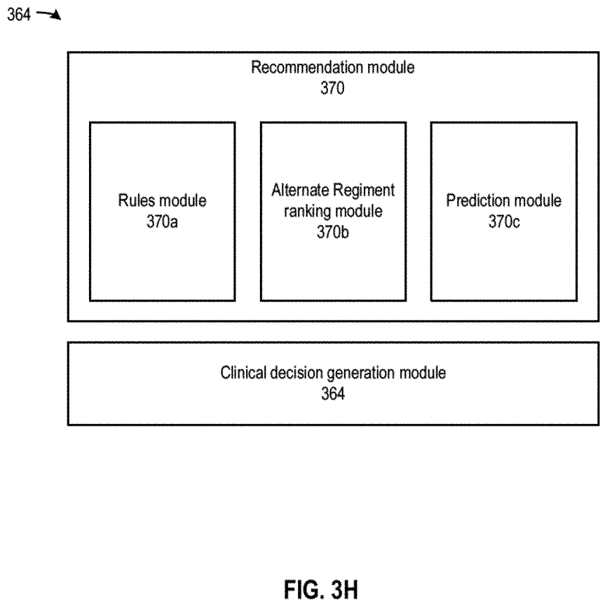

FIG. 3A illustrates an example of a digital ASP system 300 that can improve the execution of an ASP. Digital ASP system 300 can be a software system including multiple software sub-systems and modules, such as one or more ASP team subsystems 302 and one or more treating team subsystems 304. ASP team sub-system 302 can provide the ASP team with access to relevant information for a clinical decision to intervene the prescription of antibiotics and/or a diagnostic test order by the treating team, and transmit the intervention recommendation to treating team sub-system 304 (and/or other ASP team sub-systems 302) via real-time communication, which can be in the form of voice call, text messages, etc. ASP team sub-system 302 can also generate a report to record various statistics, such as the prescription of antibiotics, interventions, etc., to help administrators to evaluate the execution of the ASP. Moreover, treating team sub-system 304 can provide the treating team access to relevant information for a clinical decision to prescribe antibiotics and/or to order diagnostic tests to a patient. Treating team sub-system 304 can also receive the intervention recommendation from ASP sub-system 302, transmit a response to the intervention recommendation back to ASP sub-system 302, and transmit prescription orders (for antibiotics, diagnostic tests, etc.) to other treating team sub-systems 304 via real-time communication.

B. ASP Team Sub-System

ASP team sub-system 302 can be accessible via an ASP team interface 312, which can be a desktop interface to be provided on a display screen of a computer accessible by the ASP team, such as computers 314a and 314b. ASP team interface 312 can include an ASP data access interface 312a to provide access to the relevant information for an intervention recommendation. ASP team interface 312 can also include an ASP communication interface 312b to enable the ASP team to communicate with the treating team (or other ASP team members) about the intervention recommendation.

ASP team sub-system 302 can further include an ASP data access module 316, a patient triage module 318, an ASP intervention module 320, an ASP notification module 322, a reporting module 324, an ASP communication module 326, and an administrative module 327.

Specifically, ASP data access module 316 can be connected to one or more databases 328 over a network (not shown in the figures). Databases 328 may include, for example, a patient medical data database 328a, a guidelines database 328b, and an antibiogram database 328c. Databases 328 may further include, for example, an electronic medical record (EMR) database, a master patient index (MPI) services database, a health information exchange (HIE) server, a storage that stores image files in the format of Digital Imaging and Communications in Medicine (DICOM), a picture archiving and communication system (PACS), a laboratory information system (LIS) including genomic data, a radiology information system (RIS), an antibiogram database, and/or a hospital guideline database.

FIG. 3B and FIG. 3C illustrate examples of guidelines and antibiogram information in digital forms stored in databases 328. FIG. 3B illustrates an example of a guideline 329 in digital form. Guideline 329 can correspond to guideline 207 of FIG. 2B and can in a graph form, with each parent node (e.g., nodes 329a-g) representing a status/state of the patient's disease, whereas each leaf node (e.g., nodes 329h-k) can be associated with a list of antibiotics that can be prescribed. Guidelines database 328b can store multiple guidelines in graph form, each being associated with a particular disease. To obtain a list antibiotics that can be prescribed for a patient, ASP team sub-system 302 and/or treating team sub-system 304 can retrieve the graph of a particular guideline from guidelines database 328b based on the disease of the patient, traverse through graph by selecting the parent nodes of the guideline graph based on a state/status of the patient's disease, reach a leaf node at the end of the traversal, and obtain the list of antibiotics associated with the leaf node.

FIG. 3C illustrates an example of antibiogram data 331 in digital form. Antibiogram data 331 can include multiple antibiogram tables, each of which can contain similar information as antibiogram table 209 of FIG. 2C. Each antibiogram table can be associated with a location identifier (location ID) associated with a location where antibiotics are administered, such as ICU, regular hospital beds, nursing homes, etc. Each antibiogram table further includes multiple sections, with each section associating an antibiotic identifier of a particular antibiotic with a list of pathogens and their degrees of resistivity to the antibiotic, similar to sections 209-a and 209-b. To determine the degree of resistivity of a particular type of antibiotic for a patient, ASP team sub-system 302 and/or treating team sub-system 304 can retrieve a antibiogram table based on a location where the patient is to be administered antibiotics (e.g., ICU, regular hospital bed, nursing home, etc.), identify a section in the antibiogram table based on an identifier of the particular type of antibiotic, and obtain a list of pathogens and their degrees or resistivity. The system can further narrow down to the list of pathogens to include only pathogens that are determined to be present in the patient (e.g., based on the patient's lab result).

Referring back to FIG. 3A, ASP data access module 316 can receive a trigger to retrieve and aggregate patients data for ASP intervention determination. The trigger can be based on, for example, a command transmitted by a computer operated by the ASP team (e.g., computers 314a, 314b, etc.) to access the medical data of certain patients, a timer that indicates a review of patients' antibiotics usage/prescription by the ASP team is due, an indication (e.g., a network message from treating team sub-system 304) that a new antibiotic prescription has been entered for a patient and the prescription has not been reviewed, etc. In response to the trigger, ASP data access module 316 can obtain a list of patients whose antibiotics usage/prescription are to be reviewed by the ASP team, and transmit queries including the identifiers of the list of patients to databases 328 to obtain data of the patients. As to be discussed below, the list of patients can be determined by patient triage module 318 and can include patients who are deemed to be high priority for reviewing antibiotics usage/prescription.

ASP data access module 316 can obtain various types of data from databases 328 including, for example, various medical data of patients that are relevant for an intervention recommendation, such as their medical history, their most recent diagnoses, results of various measurements (e.g., body temperatures, blood pressures, etc.), and results of various laboratory tests (e.g., bacterial testing, culturing bacteria, antibiotic sensitivity testing, gram stain, etc.). The information may also include, for example, antibiogram information, guidelines for prescription and administering of the antibiotics, etc. Data access interface 312a can also select a subset of the aggregated data, and display the selected data in the data access interface.

1. Patient Filtering and Triage

In some examples, ASP team sub-system 302 (e.g., ASP data access module 316, ASP data access interface 312a, etc.) can automatically perform a filtering operation based on a degree of relevancy of the data to a particular patient. The degree of relevancy can be based on various data of the particular patient. For example, as described above with respect to FIG. 3C, the ASP team sub-system can automatically select, for a patient, an antibiogram table based on a location where the patient is to be administered antibiotics (e.g., ICU, regular hospital bed, nursing home, etc.) as indicated in the medical data of the patient. Moreover, based on the laboratory test results of the patient (which can indicate which pathogen is causing an infectious disease of the patient), inventory of antibiotics, etc., the ASP team sub-system can narrow down to a particular section of the antibiogram table for a particular pathogen against a narrow set of antibiotics for a particular setting, and provide the section of the antibiogram table for display in ASP data access interface 312a.

For example, referring to FIG. 3C, the antibiogram data stored in antibiogram database 328c can include multiple antibiogram tables each associated with a location of administration of the antibiotics. ASP team sub-system 302 can also determine which hospital department the patient is currently admitted to, and determine the location where the patient is most likely to be administered antibiotics. For example, if the patient is currently admitted to the ICU, ASP team sub-system 302 can determine that the patient is most likely to be administered antibiotics at the ICU, and select the antibiogram table associated with ICU.

As another example, the ASP team sub-system can also identify a particular section of a guideline (e.g., one of sections 206b-a, 206b-b, 206b-c, or 206b-d of FIG. 2B), and provide access to the identified section of the guideline via ASP data access interface 312a. Referring back to FIG. 3B, the ASP team sub-system can identify a guideline graph based on the patient's infectious disease, and traverse the guideline graph to identify parents corresponding to the kind and severity of the patient's comorbidities, etc., as indicated in the medical history of the patient. The ASP team sub-system can then output a section of the guideline associated with the identified parent nodes and including the recommended antibiotics associated with the leaf nodes at the end of traversal.

In addition, patient triage module 318 can facilitate efficient review and intervention of the patients' therapies/tests. In some examples, patient triage module 318 can automatically select a subset of patients for review of antibiotics usage/prescription. The selection can be based on, for example, the antibiotic profile, laboratory test results of the patients, medical history of the patients, intervention tracking status, etc.

Specifically, patient triage module 318 can determine a triage score for each patient, rank the patients based on their triage scores, and display a ranked list of patients in ASP team interface 312. The triage score can indicate the urgency for reviewing the patient's treatment/test. ASP team interface 312 can receive a selection from the ASP team of a particular patient in the ranked list, and display the aforementioned filtered data of the selected patient in ASP data access interface 312a.

FIG. 3D and FIG. 3E illustrate example techniques to compute the triage score of a patient. As shown in FIG. 3D, a patient may be associated with a set of attributes 330 that can be used to determine the urgency for reviewing the patient's antibiotics/diagnostic test prescription. Each attribute can also be associated with a score s0, s1, etc. In some examples, the score of an attribute can be 0 for absence of the attribute and 1 for presence of the attribute. Other score values can also be assigned. In addition, each attribute can also be associated with a weight, such as w0, w1, w2, etc. A triage score can be computed based on a weighted average of the score, with a higher score meaning it is more urgent to review the patient's antibiotics/diagnostic test prescription and vice versa.

The weights of the attributes can be determined in various ways. In some examples, the weights can be determined by the ASP team. For example, the weights can be assigned such that patients for whom new laboratory results are available, patients that warrant an active review as they now exhibit some characteristic indicating an opportunity to optimize the antibiotics they are being given (e.g., based on positive blood culture result, bug-drug mismatch showing that the current therapy is ineffective, etc.), and patients that are being administered multiple antibiotics, broad-spectrum drug, restricted antibiotics, or antibiotics on the shortage list, can receive review by the ASP team ahead of other patients who do not have these features. In some examples, the weights of the attributes can also be determined automatically by patient triage module 318 based on other criteria, such as a history of interventions. For example, if the ASP team has intervened the prescription of antibiotics/diagnostics tests for a patient having certain attributes before, patient triage module 318 can automatically increase the weights of those attributes.

In addition, patient triage module 318 can rank the patients based on other criteria. For example, as to be described below, ASP notification module 322 can generate a notification if certain attributes of the patient, based on the latest laboratory test results and/or prescriptions, indicate that the patient warrants review from the ASP team. Referring to FIG. 3 F, ASP notification module 322 may generate a notification if, for example, the latest patient's medical data indicate bug/drug mismatch, redundant antibiotics coverage, positive culture, allergy to current/prescribed therapy, drug-lab mismatch, and/or intravenous (IV) to oral (PO) opportunity, etc. Patient triage module 318 can count a number of notifications ASP notification module 322 has generated for each patient since the last time the patient's prescription was reviewed via ASP team sub-system 302, and rank the patients based on the number of notifications. Given that a notification can be generated when a new laboratory test result and/or prescription warrants review, a higher number of notifications generated for the patient may indicate that it is more urgent to review the patient's antibiotics prescription. Therefore, patient triage module 318 can rank the patients based on the notifications, with the patient having the highest number of notifications ranked the highest.

In some examples, patient triage module 318 can also rank the patients based on the prescription of antibiotics. For example, patient triage module 318 can compute, for each patient, a composite antibiotics score that reflects the characteristics of the antibiotics currently prescribed to the patient, and then rank patients having the same number of notifications based on the composite antibiotics score. The ranking based on composite antibiotics scores can be a secondary ranking after the number of notifications. FIG. 3E illustrates an example antibiotics score table 332 and an example ranking of patients 334. As shown in antibiotics score table 332, an antibiotic can be assigned a score that reflects whether it is a broad spectrum antibiotics that treats a broad range of pathogens, whether it is in shortage, whether it is restricted, etc. A restricted antibiotic can be assigned the highest score, which indicates the highest priority for review of its prescription. Each antibiotic prescribed to a patient can then be assigned a score based on table 332, and the scores for a patient can be summed to generate an antibiotic composite score. Referring to example ranking 334, patients can be ranked based on the number of notifications, with patients having the highest number of notifications being ranked the highest (e.g., patients A and B). Among patients (e.g., patients A and B) having the same number of notifications, the patients can be further ranked based on their composite antibiotics scores, with the patient having the highest composite antibiotic score ranked the highest.

In example ranking 334, patient triage module 318 can also rank the patients based on a duration in which the patients are administered a particular antibiotic. A patient who has been administered an antibiotic for a large number of consecutive days can be ranked higher than another patient who has been administered the antibiotic (or other antibiotics) for a fewer number of consecutive days. In some examples, the ranking based on the antibiotic administration duration can be performed on a group of patients having the same number of notifications and composite antibiotics scores (e.g., patients C and D) as a tertiary ranking.

2, ASP Intervention

Referring back to FIG. 3A, in addition to aggregating and displaying the relevant data for an intervention recommendation, ASP team sub-system 302 further includes ASP intervention module 320 to output the intervention recommendation. ASP intervention module 320 can accept an input from an ASP team member, via ASP team interface 312, and generate an intervention recommendation based on the input. ASP intervention module 320 can then send the intervention recommendation to ASP notification module 322, which can generate a notification/text message including the intervention recommendation and transmit the notification to treating team sub-system 304 via ASP communication module 326.

In some examples, ASP sub-system 302 can be accessible via a mobile device. In such examples, ASP notification module 322 can output the notification on an idle screen of the mobile device and, upon detecting a selection of the notification, switch the mobile device from an idle state to an active state and activate treating tam interface 360. Treating team notification module 366 can also receive a clinical decision about a prescription order by treating team clinical decision module 364, and transmit the clinical decision as notifications to other treating team sub-systems 304.

FIG. 3F illustrates examples of internal components of ASP intervention module 320. As shown in FIG. 3F, ASP intervention module 320 can include a recommendation module 340, an intervention recommendation generation module 342, and a tracking module 344. Recommendation module 340 can generate a recommendation for an intervention recommendation, and output the recommendation to ASP team interface 312. The intervention recommendation may include, for example, dosing changes, escalating/deescalating antimicrobials, conversion from IV to PO, etc. In some examples, recommendation module 340 can output the recommendation to ASP data access interface 312a, which can display the recommendation together with the medical data, guidelines, and/or antibiogram information that support the recommendation.

Intervention recommendation generation module 342 allows the ASP team member to create an intervention recommendation for the treating team based on, for example, manually filling a form provided in ASP team interface 312 (e.g., based on viewing the recommendation), or based on selecting a recommendation (to the ASP team) provided by recommendation module 340 to automatically generate an intervention recommendation, and then output the intervention recommendation to ASP notification module 322. ASP notification module 322 can then transmit the intervention recommendation as a notification/text message to treating team sub-system 304. Tracking module 344 can track the status (compliance or non-compliance) of an intervention recommendation based on, for example, monitoring for a response from team sub-system 304, or an input from the ASP team member via ASP team interface 312.

Recommendation module 340 can generate the recommendation (for the ASP team) to generate an intervention recommendation (to the treating team) based on various techniques. As shown in FIG. 3F, recommendation module 340 can include a rules module 340a which can generate a recommendation based on applying one or more rules on the medical data. The rules may indicate, for example, selecting one or more antibiotics that are effective to treat a patient's disease while minimizing pathogen resistance to the selected antibiotics. The rules can be applied to the patient's medical data, guidelines, and antibiogram information to generate a recommendation. The medical data can include, for example, a disease of the patient, clinical response, diagnostic test results (e.g. cultures and susceptibility testing), and other lab parameters (e.g., creatinine, drug levels).

As an example, referring back to FIG. 3B, based on a disease of the patient as indicated in the medical data of the patient, rules module 340a can retrieve a guideline graph from guidelines database 328b. Moreover, rules module 340a can traverse the guideline graph based on a state of the disease of the patient (e.g., whether or not the patient is admitted to the hospital, a degree of severity, presence/absence of comorbidities, etc.) and identify a list of candidate antibiotics that can be prescribed to the patient as a treatment for the disease, as well as the dosages of the candidate antibiotics, at the end of the traversal. Rules module 340a can determine whether the antibiotics prescribed to the patient are included in the list of recommended antibiotics, and whether the dosages of the prescribed antibiotics match the recommended dosages. If the prescribed antibiotics are not in the list of recommended antibiotics, or that the prescribed dosages do not match the recommended dosage, rules module 340a may determine a recommendation for intervening the antibiotics prescription (e.g., changing to a different antibiotic, changing the dosage, etc.).

In addition, referring back to FIG. 3C, rules module 340a can retrieve an antibiogram table based on a treatment location of the patient, and identify sections of the antibiogram table corresponding to the antibiotics being prescribed. In each section, rules module 340a can determine the degree of resistance of one or more pathogens that cause the patient's disease (as indicated in, for example, the lab test results of the patient) to the prescribed antibiotics. If the degree of resistance exceeds a particular threshold, rules module 340a may also determine a recommendation for intervening the antibiotics prescription.

Moreover, recommendation module 340 can also include an alternate regiment ranking module 340b that can compute a benefit score and a risk score for each alternative antibiotic therapy regiment, which can include one or more different antibiotics, rank the alternate regiments based on a ratio between the benefit score and the risk score for each regiment, and select the regiment having the highest ratio as the intervention recommendation. The benefit score can be based on, for example, a susceptibility of the pathogen to the antibiotics, whereas the risk score can be based on, for example, whether any of the antibiotics in the regiment is redundant, a risk of the pathogen becoming resistant to a particular antibiotic in the regiment, etc. The risk score can also be computed based on medical history of the patient (e.g., whether the patient has experienced resistance), suspected diagnosis, etc. By taking the risk into account, it can avoid a scenario where a broad-spectrum antibiotic is always recommended by recommendation module 340 since it will cover most types of pathogens.

In addition, recommendation module 340 can also include a prediction module 340c, which can generate a recommendation based on performing a prediction operation. The goal of the prediction operation can be to predict the likelihood/probability that a patient is on an inappropriate antibiotic therapy, or that intervention is needed. The prediction can be based on the antibiotic treatment received by other patients. For example, if a patient receives a certain antibiotic for five days, whereas other patients having the same infectious disease receive the same antibiotic for only two days and recover, recommendation module 340 may predict that the patient receives an inappropriate antibiotic therapy that has exceeded an expected duration (two days).

Recommendation module 340 can process the medical data of a patient and determine whether to transmit a recommendation for ASP intervention based on receiving a trigger. The trigger may be the same trigger that causes ASP data access module 316 to retrieve and aggregate patients data for ASP intervention determination, or a different trigger based on, for example, an indication from databases 328 that certain new medical data of the patient (e.g., lab test results, or other new medical data that have not been processed by the sub-system) are available. The indication can be in the form of a network message transmitted by the databases in response to a query transmitted by recommendation module 340 when ASP data access interface 312a displays the medical data of the patient.

As an example, in response to ASP data access interface 312a displaying the medical data of a patient after ASP data access interface 312a receives a selection of the patient by the ASP team member, recommendation module 340 may transmit a query to databases 328 for updates (if any) of the medical data of the patient. If recommendation module 340 detects that certain new medical data relevant for an ASP intervention decision (e.g., lab test results, such as Gram negative rod (GNR) results) becomes available, detects a request to generate a recommendation, and/or detects the expiration of a timer indicating that a review of the patient's antibiotics prescription is due, recommendation module 340 can process the medical data of the patient and determine whether to generate and transmit a recommendation for ASP intervention.

3. ASP Notification

Referring back to FIG. 3A, ASP notification module 322 can generate a notification, which can be sent to treating team sub-system 304 or other modules within ASP team sub-system 302. FIG. 3G illustrates examples of internal components of ASP notification module 322, which can include a rule-based notification module 352 and an intervention notification module 354. Rule-based notification module 352 can generate a notification based on one or more rules. For example, as described with respect to FIG. 3H, rule-based notification module 352 may access databases 328, or information aggregated by ASP data access module 316, and generate a notification (e.g., system-based notification 356a) if, for example, the latest patient's medical data indicate bug-drug mismatch, redundant antibiotics coverage, positive culture, allergy to current/prescribed therapy, drug-lab mismatch, and/or intravenous (IV) to oral (PO) opportunity, etc., based on applying the guidelines onto the antibiogram information and latest medical data of the patient. As another example, rule-based notification module 352 may generate a notification based on availability of new medical data of the patient (e.g., new blood test results, new blood culture results, etc.). The notification can be sent to treating team sub-system 304, and patient triage module 318 may determine a ranking of patients based on the number of notifications shown in FIG. 3E. In addition, intervention notification module 354 can receive an intervention recommendation from intervention recommendation generation module 342, which can be either generated either by intervention recommendation generation module 342 or receiving a selection, by the ASP team, of a recommendation from recommendation module 340. Intervention recommendation generation module 342 can then generate either a manual intervention notification 356b (from intervention recommendation module 342), or a recommendation based intervention notification 356c (from recommendation module 340).

Referring back to FIG. 3A, ASP team sub-system 302 can include reporting module 324 and ASP communication module 326. Reporting module 324 can generate a report to record various statistics, such as the days of antibiotics therapy with different parameters, interventions (compliance or non-compliance, based on outputs of tracking module 344), number of infections per 1000 patient days or per 100 admission, etc., at a particular hospital. The report can also include comparative data reports for different hospitals. In some examples, the report may include a report on days of therapy, an intervention acceptance report, etc. In some examples, reporting module 324 can also transmit the report to an external agency, such as National Healthcare Safety Network (NHSN) of Centers for Disease Control and Prevention (CDC).

In addition, ASP communication module 326 can provide real-time communication among ASP team sub-systems 302 and treating team sub-systems 304. The real-time communication can be in the form of, for example, voice call, text messages, etc. Notifications can be sent from ASP notification module 322 to other ASP team sub-systems 302 and treating team sub-systems 304 via ASP communication module 326. ASP communication module 326 can be accessible via ASP communication interface 312b. In some examples, ASP communication interface 312b can be in the form of a text-messaging interface, in which the notification, as well as a response to the notification from the treating team sub-system, can be displayed in the form of text messages. ASP team sub-system 302 can display ASP data access interface 312a and ASP communication interface 312b concurrently within ASP team interface 312, which allows the users to communicate via text messages or voice while having access to the medical data, to facilitate the collaboration experience.

In addition, ASP team sub-system 302 can also include administrative module 327, which can support various administrative functions, such as configuring the generation of notifications, setting access rights to the notification (e.g., which member of the ASP team can generate/receive notification, which member of the treating team can receive a notification from the ASP team sub-system via the treating team sub-system, etc.), editing/abstracting the medical data (e.g., editing of the guidelines, converting the data into proprietary formats, etc.).

C. Treating Team Subsystem

Reference is now made to treating team sub-system 304, which can provide the treating team access to relevant information for a clinical decision to prescribe antibiotics and/or diagnostic tests to a patient. Treating team sub-system 304 can also receive the intervention recommendation from ASP sub-system 302, transmit a response to the intervention recommendation back to ASP sub-system 302, and transmit prescription orders (for antibiotics, diagnostic tests, etc.) to other treating team sub-systems 304 via real-time communication.

Specifically, treating team sub-system 304 can be accessible via a treating team interface 360, which can be a mobile interface provided on a mobile device (e.g., smart phone, tablet, laptop computer, etc.) of a member of the treating team, such as mobile devices 361a and 361b. Treating team interface 360 can include a treating team data access interface 360a to provide access to the relevant information for prescribing antibiotics (e.g., first dose, empiric therapy, etc.) and/or ordering diagnostic tests related to a bacterial infection (e.g., staining and examination, culture, testing of pathogen's susceptibility/sensitivity to antibiotics, etc.) to a patient. Treating team interface 360 further includes a treating team communication interface 360b to enable a treating team member to communicate with other treating team members (e.g., pharmacists) about the prescription. Treating team communication interface 360b also enables the treating team member to access a notification from ASP team sub-system 302, which can include system-generated notification 356a, intervention notifications 356b and 356c, etc., of FIG. 3G.

Treating team sub-system 304 can further include a treating team data access module 362, a treating team clinical decision module 364, a treating team notification module 366, and a treating team communication module 368.

1. Treating Team Data Access

Similar to ASP data access module 316 of ASP team sub-system 302, treating team data access module 362 of treating team sub-system 304 can source the relevant information to support a decision of prescribing certain antibiotics and/or ordering certain diagnostic tests from databases 328, and provide the information to treating team data access interface 360a. Treating data access module 362 can retrieve and aggregate the data from databases 328 based on receiving a trigger, such as a selection via treating team data access interface 360a (by the treating team) to review the medical history of one or more patients. Based on receiving the selection, treating team data access module 362 can transmit the identifiers of the patient to databases 328 to retrieve the medical data of that patient.

Treating team data access module 362 can source various types of data including, for example, various medical data of patients that are relevant for a recommendation to prescribe an antibiotics and/or a diagnostic test, such as their medical history, their most recent diagnoses, results of various measurements (e.g., body temperatures, blood pressures, etc.), and results of various laboratory tests (e.g., bacterial testing, culturing bacteria, antibiotic sensitivity testing, gram stain, etc.). The information may also include, for example, antibiogram information, guidelines for prescription and administering of the antibiotics, etc. Treating team data access interface 360a can also select a subset of the aggregated data, and display the selected data in the data access interface.

In some examples, treating team sub-system 304 (e.g., treating team data access module 362, treating team data access interface 360a, etc.) can automatically perform a filtering operation based on a degree of relevancy of the data to a particular patient, in similar ways as ASP team sub-system 302. For example, the antibiogram information can include multiple sections, each associated with a location of administration of the antibiotics. Treating team sub-system 304 can determine which hospital department the patient is currently admitted to, and determine the location where the patient is most likely to be administered antibiotics. For example, if the patient is currently admitted to the ICU, treating team sub-system 304 can determine that the patient is most likely to be administered antibiotics at the ICU, and select the section of the antibiogram information associated with ICU. In some examples, the filtering operation can also be performed based on inputs (e.g., a query) from the user.

2. Treating Team Clinical Decision

In addition, treating team sub-system 304 can further include treating team clinical decision module 364 to output a clinical decision of prescribing certain antibiotics and/or ordering certain diagnostic tests. Treating team clinical decision module 364 can accept an input from a treating team member, via treating team interface 360, and generate the clinical decision based on the input. Treating team clinical decision module 364 can then send the clinical decision to treating team notification module 366, which can generate a notification and send the notification to other treating team sub-systems 304 via treating team communication module 368.

FIG. 3H illustrates examples of internal components of treating team clinical decision module 364. As shown in FIG. 3H, treating team clinical decision module 364 can include a recommendation module 370 and a clinical decision generation module 372. Recommendation module 370 can generate a recommendation for a clinical decision of prescribing certain antibiotics and/or ordering certain diagnostic tests, and output the recommendation in treating team interface 360. The recommendation may include, for example, the prescription of one or more antibiotics, method of administering the antibiotics, dose, and/or duration of the therapy. In some examples, recommendation module 370 can output the recommendation to treating team data access interface 360a, which can display the recommendation together with the medical data, guidelines, and/or antibiogram information that supports the recommendation.

Clinical decision generation module 372 allows the treating team member to input an intervention recommendation based on, for example, manually filling a form provided in treating team interface 360 (e.g., based on viewing the recommendation), or based on selecting a recommendation provided by recommendation module 370 to automatically generate a clinical decision, and then output the clinical decision to treating team notification module 366.

Recommendation module 370 can generate the recommendation for the clinical decision based on various techniques. As shown in FIG. 3H, recommendation module 370 can include a rules module 370a which can generate a recommendation based on applying one or more rules on the guidelines, the antibiogram information, and the medical data. Similar to rules module 340a for ASP team sub-system 302, the rules may indicate, for example, selecting one or more antibiotics that are effective to treat a patient's disease while minimizing pathogen resistance to the selected antibiotics. The medical data can include, for example, clinical response, diagnostic test results (e.g. cultures and susceptibility testing), and other lab parameters (e.g., creatinine, drug levels).

As an example, referring back to FIG. 3B, based on a disease of the patient as indicated in the medical data of the patient, rules module 340a can retrieve a guideline graph from guidelines database 328b. Moreover, rules module 340a can traverse the guideline graph based on a state of the disease of the patient (e.g., whether or not the patient is admitted to the hospital, a degree of severity, presence/absence of comorbidities, etc.) and identify a list of candidate antibiotics that can be prescribed to the patient as a treatment for the disease, as well as the dosages of the candidate antibiotics, at the end of the traversal.

In addition, referring back to FIG. 3C, rules module 370a can retrieve an antibiogram table based on a treatment location of the patient, and identify sections of the antibiogram table corresponding to the list of recommended antibiotics. In each section, rules module 370a can determine the degree of resistance of one or more pathogens that cause the patient's disease (as indicated in, for example, the lab test results of the patient) to the prescribed antibiotics. Rule module 370a can rank the recommended antibiotics based on the degrees of resistance, and include a subset of the list of recommend antibiotics having the lowest degrees of resistance, or having degrees of resistance below a threshold, in the recommendation.

Moreover, recommendation module 370 can also include an alternate regiment ranking module 370b that can compute a benefit score and a risk score for each alternative antibiotic therapy regiment, which can include one or more different antibiotics, rank the alternate regiments based on a ratio between the benefit score and the risk score for each regiment, and select the regiment having the highest ratio as the intervention recommendation. The benefit score can be based on, for example, a susceptibility of the pathogen to the antibiotics, whereas the risk score can be based on, for example, whether any of the antibiotics in the regiment is redundant, a risk of the pathogen becoming resistant to a particular antibiotic in the regiment, etc. The risk score can also be computed based on a medical history of the patient (e.g., whether the patient has experienced resistance), suspected diagnosis, etc.

In addition, recommendation module 370 can also include a prediction module 370c, which can generate a recommendation based on performing a prediction operation. The goal of the prediction operation can include, for example, predicting when an antibiotic treatment is needed. The prediction can be based on determining the likelihood that a bacterial infection requires an antibiotic treatment versus the likelihood that the infection is a self-resolving infection, or that the disease of a patient is a non-infectious process. In some examples, the prediction can be based on a trade-off between “number needed to treat” (how many patients need to be treated with an antibiotic in order to benefit one patient?) versus “number needed to harm” (how many patients could be treated with an antibiotic before one experiences a treatment harm?).

Recommendation module 370 can process the medical data of a patient and determine whether to transmit a recommendation for a clinical decision of prescribing certain antibiotics and/or ordering certain diagnostic tests based on receiving a trigger. The trigger may be based on the same trigger as the trigger that causes treating team data access interface 360a to retrieve and aggregate the data from databases 328, or can be based on a different trigger based on, for example, the patient's medical data being displayed in treating team data access interface 360a, a command from the treating team member, an indication from databases 328 that certain new medical data of the patient (e.g., lab test results) are available, etc.

For example, in response to treating team data access interface 360a displaying the medical data of a patient, recommendation module 370 may transmit a query to databases 328 for updates (if any) of the medical data of the patient. If recommendation module 370 detects that certain new medical data relevant for a clinical decision of prescribing certain antibiotics and/or ordering certain diagnostic tests (e.g., lab test results) becomes available, recommendation module 370 can process the updated medical data of the patient and determine the recommendation for a clinical decision.

3. Communication Between Treating Team and ASP Team

Referring back to FIG. 3A, treating team sub-system 304 can further include treating team notification module 366 and treating team communication module 368. Treating team notification module 366 can handle both reception and transmission of notifications. As described above, treating team sub-system 304 can receive a notification from ASP team sub-system 302, which can include system-generated notification 356a, intervention notifications 356b and 356c, etc., of FIG. 3G. Treating team notification module 366 can output the notification on an idle screen of the mobile device and, upon detecting a selection of the notification, switch the mobile device from an idle state to an active state and activate treating team interface 360. In some examples the notification can be in the form of a snoozing notification so that the treating team can act on the notification at a later time. Treating team notification module 366 can also receive a clinical decision output by treating team clinical decision module 364 and transmit the clinical decision as notifications to other treating team sub-systems 304.

In addition, treating team communication module 368 can provide real-time or asynchronous communication with ASP communication module 326 of ASP team sub-systems 302, and with another treating team communication module 368 of another treating team sub-system 304. As described above, the real-time communication can be in the form of, for example, voice call, text messages, etc. Notifications, as well as responses from the treating team to intervention recommendations from the ASP team, can be sent/received via treating team communication module 368. Treating team communication module 368 can also be accessible via treating team communication interface 360b. In some examples, treating team communication interface 360b can also be in the form of a text-messaging interface, in which the notification, as well as a response to the notification from the treating team sub-system, can be displayed in the form of text messages. Treating team sub-system 304 can display treating team data access interface 360a and treating team communication interface 360b concurrently within treating team interface 360, which allows the users to communicate via text messages or voice while having access to the medical data, to facilitate the collaboration experience.

### III. Examples of Interfaces Provided by a Digital ASP System

A. Examples of ASP Team Interface

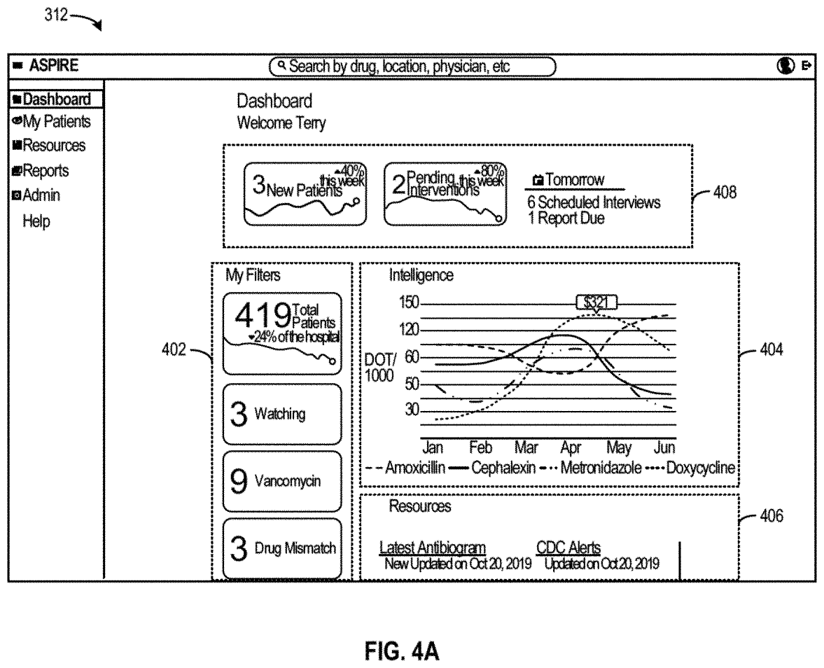

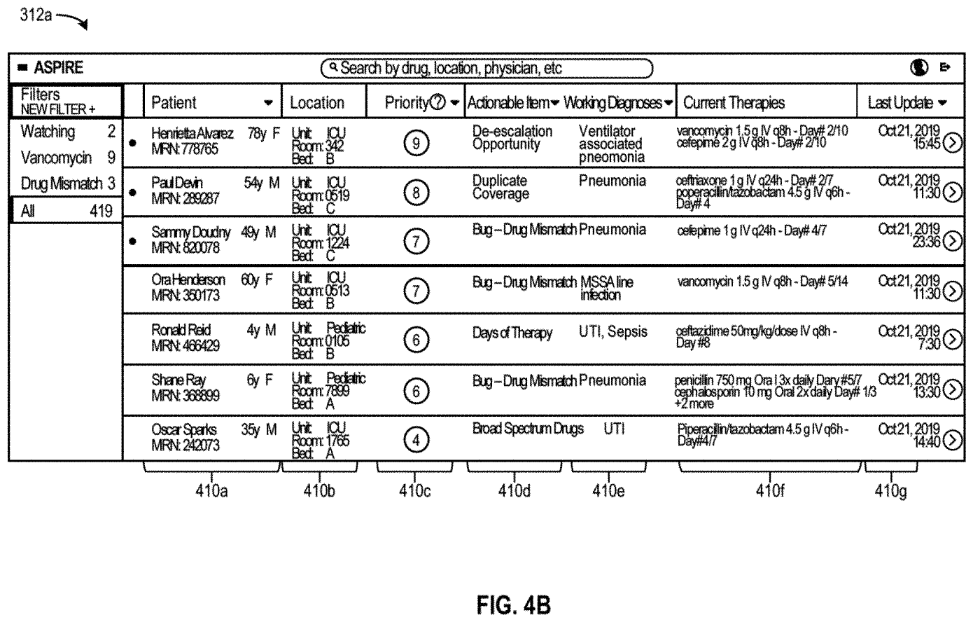

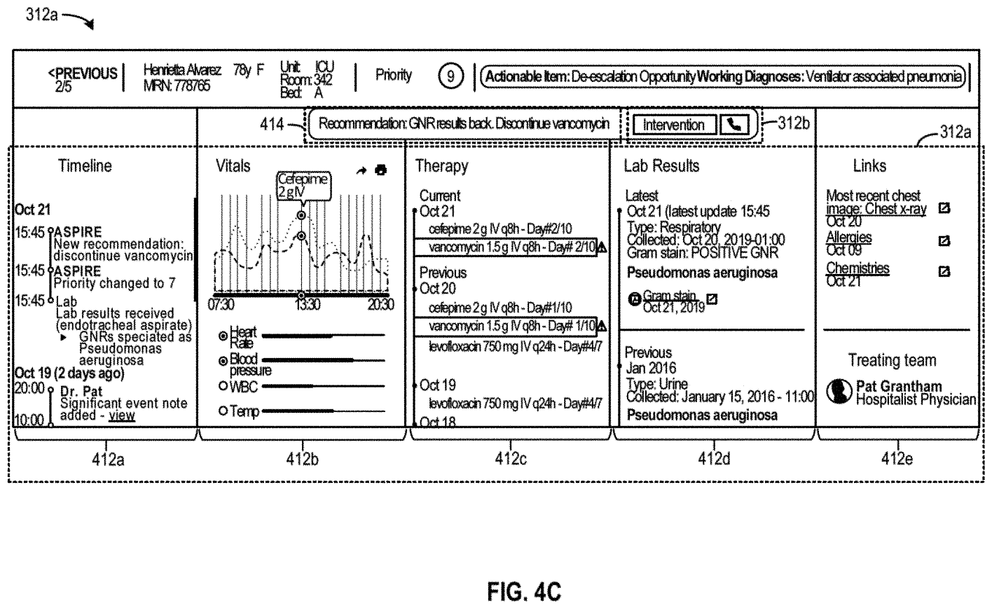

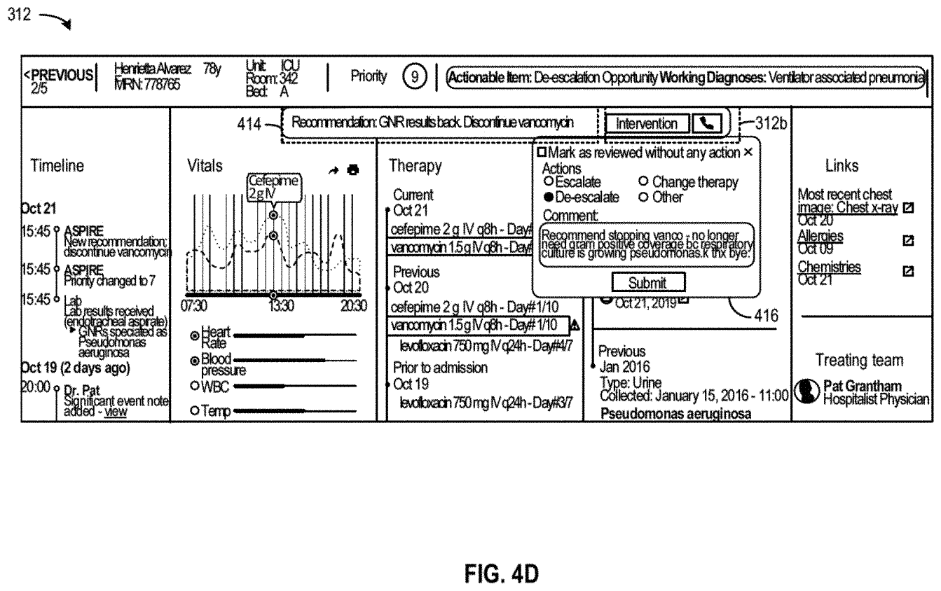

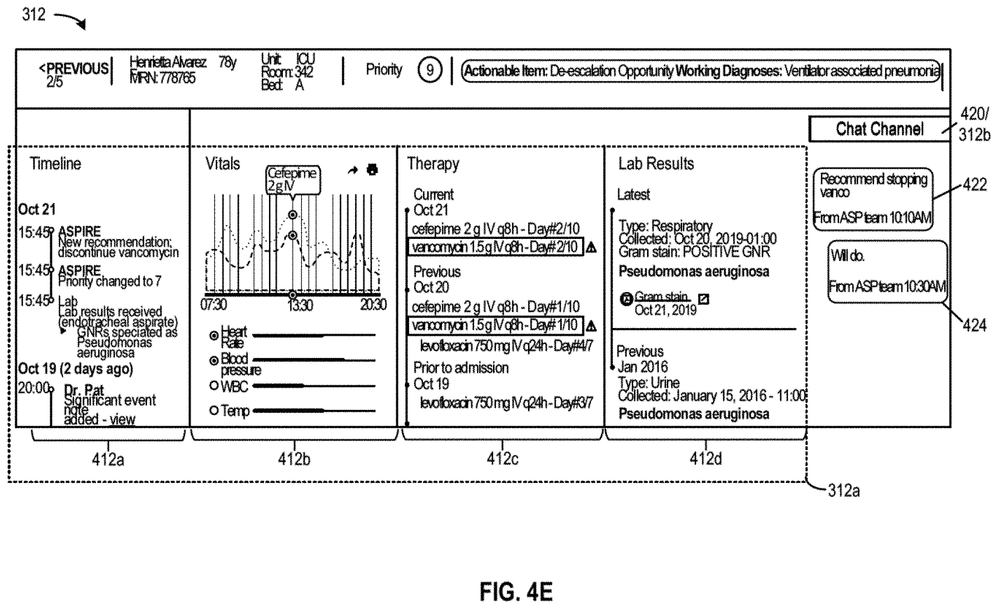

FIG. 4A-FIG. 4E illustrate examples of ASP team interface 312. FIG. 4A illustrates that ASP team interface 312 shows a dashboard interface from which the ASP team member can access different components of ASP team sub-system 302. For example, the dashboard interface includes a section 402 to access a filtered list of patients to be reviewed and a summary of the filter (e.g., patients showing drug mismatch or being prescribed with the antibiotic vancomycin are included). The dashboard interface further includes a section 404 to access a report provided by reporting module 324, a section 406 including links to resources accessible by the ASP team member (e.g., latest antibiogram, guidelines, alerts, etc.), and a section 408 indicating scheduled interventions and reports.

FIG. 4B illustrates another example of ASP team interface 312, which can be displayed upon detecting a selection of section 402 in the dashboard interface of FIG. 4A. As shown in FIG. 4B, ASP data access interface 312a can display a filtered listing of patients. The list includes, for each patient, biography information 410a, treatment location 410b, priority for review 410c, actionable item(s) 410d, diagnosis 410e, current antibiotics therapy 410f, and last updated date 410g. The patients are listed according to priority 410c, which can be determined by patient triage module 318. Actionable item(s) 410d can be based on system-generated notifications 356a of FIG. 3G which can indicate intervention opportunities.

Each patient in the filtered listing of FIG. 4B is selectable to review the patient's data, which can be displayed in ASP data access interface 312a. FIG. 4C-FIG. 4E illustrates an example of ASP data access interface 312a and ASP communication interface 312b for reviewing the antibiotic therapy of a selected patient. As shown in FIG. 4C, ASP data access interface 312a can display various types of data aggregated by ASP data access module 316 including, for example, a timeline 412a including a history of events of the patient on ASP team sub-system 302 (e.g., a history of recommendations, interventions, arrival of laboratory test results, etc.), vitals 412b (e.g., heart rate, blood pressure, temperature, etc.) of the patient, a history of antibiotic therapies 412c received by the patient, and a history of laboratory test results 412d of the patient. All these data can be part of the medical data of the patient and retrieved from patient medical data database 328a of FIG. 3A. In addition, ASP data access interface 312a also displays selectable icons 412e to provide access to other medical data (e.g., chest x-ray, allergies, etc.) of the patient. All these information are relevant, or even needed, to generate an intervention recommendation, and all these information are accessible/displayed to the ASP team member in a single data access interface. Compared with a case where the ASP team member needs to procure this information from different sources or look up this information in different interfaces or from different devices, such arrangements can substantially improve the ASP team's access to the relevant/necessary information for an intervention recommendation, which in turn allows the ASP team to generate a correct intervention recommendation quickly.

In addition, ASP team interface 312 also displays a recommendation 414 for intervention generated by recommendation module 340 of ASP intervention module 320. Recommendation 414 can be generated based on receiving the latest Gram negative rod (GNR) results and applying the rules on the GNR result. In the example of FIG. 4C, recommendation 414 suggests an intervention to discontinue the usage of vancomycin based on the GNR results. ASP team interface 312 also displays ASP communication interface 312b which provides the options of calling or sending a recommendation-based notification to communicate the intervention recommendation. Referring to FIG. 4D, upon receiving a selection of sending a recommendation based notification, ASP communication interface 312b can display a form 416 to allow the user to customize the notification, prior to sending the notification.

FIG. 4E illustrates another example of ASP communication interface 312b. As shown in FIG. 4E, ASP communication interface 312b can be in the form of a text-messaging interface 420 in which a recommendation-based notification 422 (generated from recommendation 414) and a response message 424 from the treating team are displayed, which allow real-time communication to take place between the ASP team and the treating team.

B. Examples of a Treating Team Interface or an ASP Team Interface on a Mobile Device

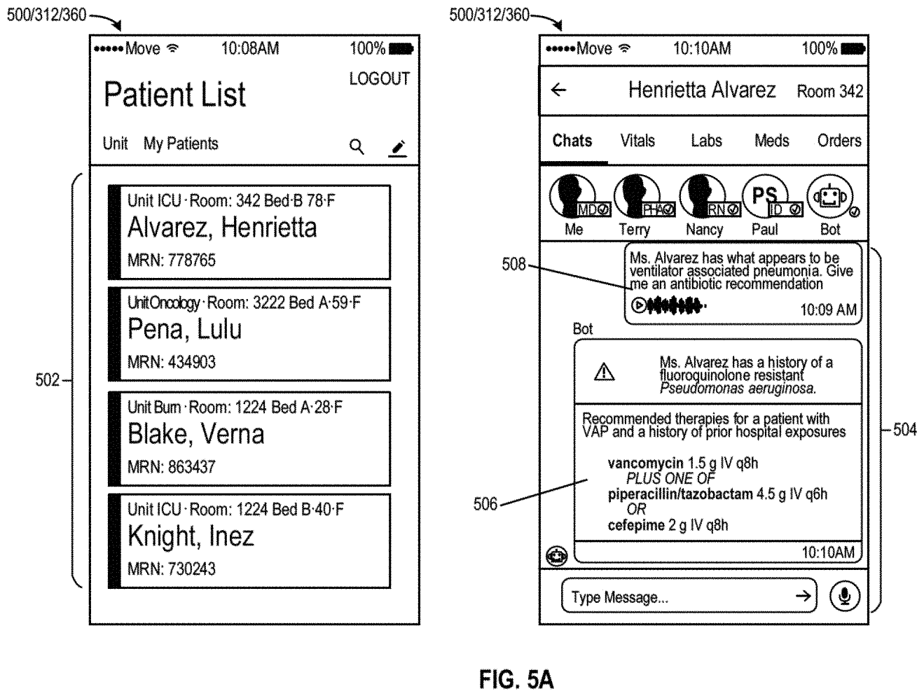

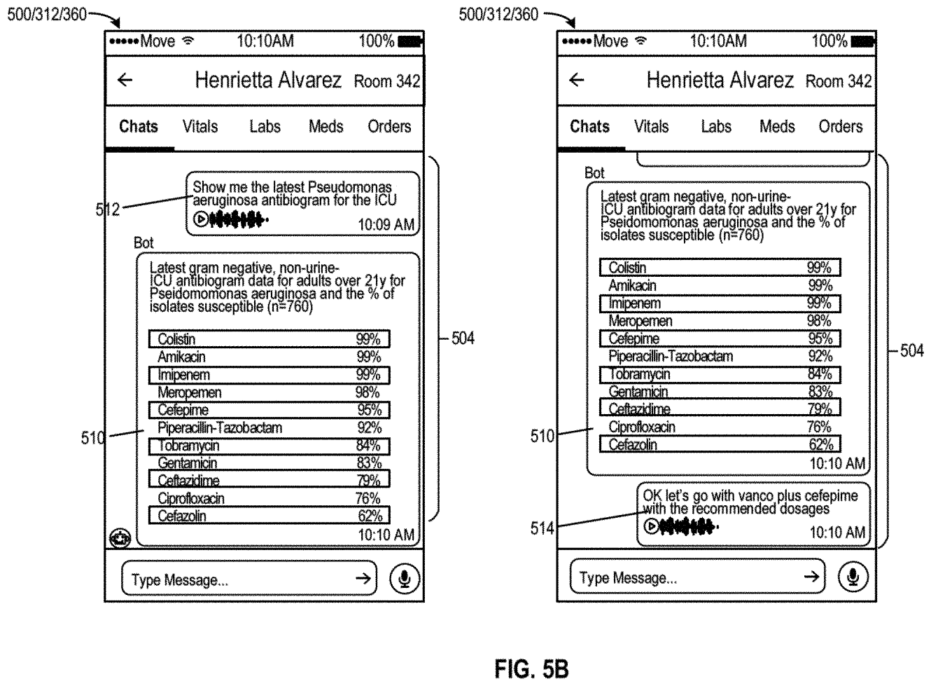

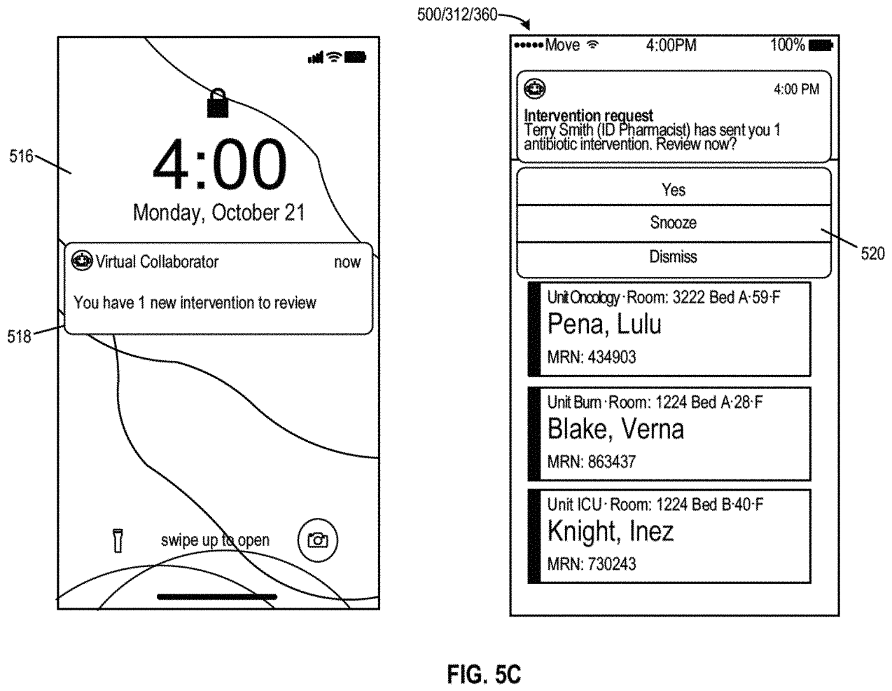

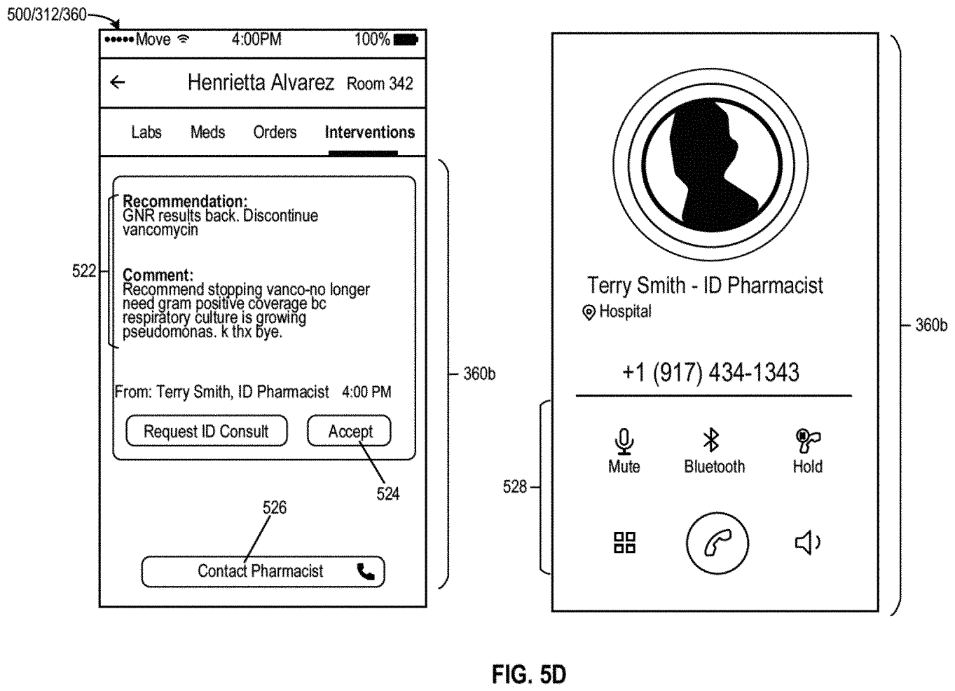

FIG. 5A-FIG. 5D illustrate examples of a mobile interface 500, which can be treating team interface 360 or a mobile version of ASP team interface 312. On the left of FIG. 5A, interface 500 shows a listing of patients 502. In a case where interface 500 is a mobile version of ASP team interface 312, the patients can be ranked by patient triage module 318. Each patient is selectable by the treating team member to show the patient's medical data and to send a clinical decision (e.g., antibiotics therapy). Upon receiving a selection of one of the patients, ASP data access module 316 or treating team data access module 362 can retrieve the medical data of the selected patient from databases 328.

On the right of FIG. 5A, a combined interface 504 is shown including treating team data access interface 360a (or ASP data access interface 312a) and treating team communication interface 360b (or ASP communication interface 312b). As shown on the right FIG. 5A, a recommendation 506 can be generated by recommendation module 370 of treating team clinical decision module 364 (or recommendation module 340 of ASP intervention module 320), upon receiving a request 508 by the treating team member.

FIG. 5B illustrates another example of mobile interface 500. As shown on the left of FIG. 5B, mobile interface 500 can output an antibiogram chart 510 that is specific for ICU, for adults of age 21 or over, and for a particular pathogen Pseidomomonas aeruginosa, upon receiving a query 512. The amount of information included in antibiogram chart 510 can be selected by treating team data access module 362 of treating team sub-system 304 (or data access module 316 of ASP team sub-system 302) based on query 512 as well as the criteria, such as past queries, a degree of relevancy, etc. For example, treating team data access module 362 can select the antibiotics to which the pathogen Pseidomomonas aeruginosa has the highest susceptibility and include those in antibiogram chart 510, to provide as much relevant antibiogram information within the limited screen size of a mobile device, rather than the antibiogram table 209 of FIG. 2C which may contain sections of information not relevant for a particular patient. On the right of FIG. 5B, treating team communication interface 360b can receive a message 514 including a clinical decision from the treating team member. Treating team clinical decision module 364 can receive message 514 and forward the message to treating team notification module 366, which can then generate a notification and send the notification to another treating team sub-system 304 operated by another treating team member (e.g., a pharmacist).

FIG. 5C and FIG. 5D illustrate examples of operations when treating team notification module 366 of treating team sub-system 304 (or ASP notification module 322 of ASP team sub-system 302) receives a notification, such as an intervention notification, from ASP team sub-system 302. As shown on the left of FIG. 5C, a mobile device can be in an idle state and a home screen 516 is displayed. Upon receiving a notification 518, the mobile device can display notification 518 in home screen 516 as a push notification. Notification 518 is selectable to cause the mobile device to switch from an idle state to active state, in which the mobile device can display training team interface 312 as well as a prompt 520 about whether to review notification 518. Referring to the left of FIG. 5D, upon detecting that the user selects to review notification 518, treating team communication interface 360b can display intervention recommendation 522 included in notification 518, as well as selectable icons 524 (e.g., to accept the intervention recommendation) and 526 (to call the sender of notification 518) for responding to the intervention recommendation. Referring to the right of FIG. 5D, upon detecting the selection of icon 526, treating team communication interface 360b can provide a dial screen 528 to enable the treating team member to make a phone call to the sender.

### IV. Method

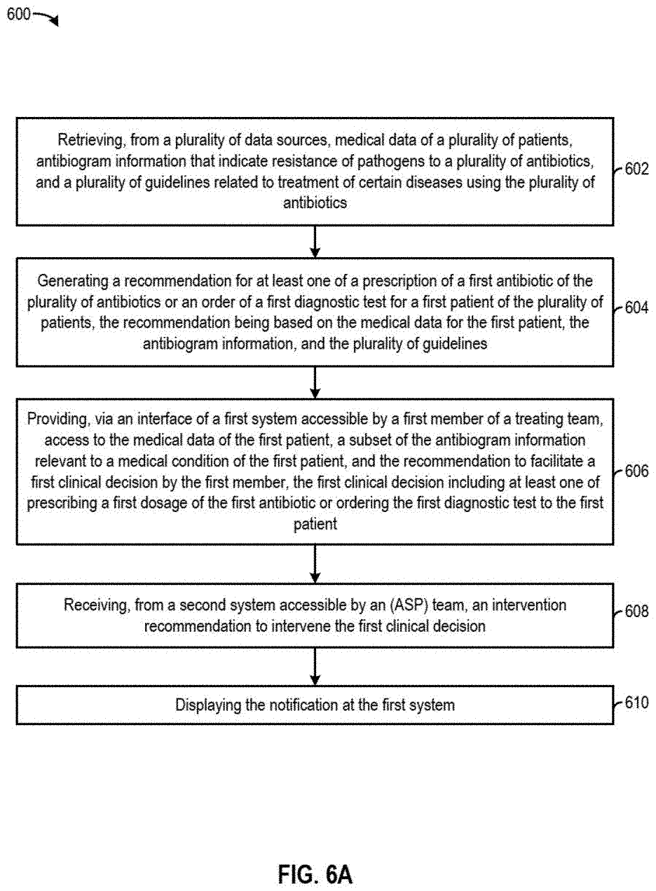

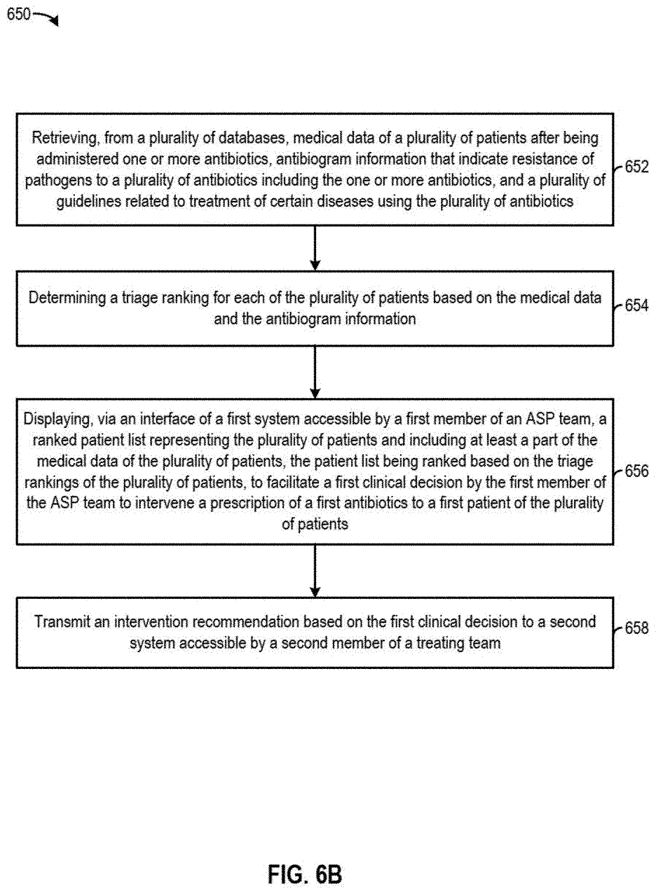

FIG. 6A and FIG. 6B illustrate examples of methods to support an antimicrobial stewardship program. The methods can be implemented by a digital ASP system, such as digital ASP system 300.

A. Example Method to be Performed by a Treating Team Sub-System

FIG. 6A illustrates an example of a method 600 that can be implemented by, for example, treating team sub-system 304.

In step 602, treating team sub-system 304 can aggregate, from a plurality of databases, medical data of a plurality of patients, characteristics information of a plurality of antibiotics, and a plurality of guidelines related to treatment of certain diseases using the plurality of antibiotics.

For example, treating team data access module 362 of treating team sub-system 304 can aggregate various types of medical data from one or more databases 328, such as an electronic medical record (EMR) database, a master patient index (MPI) services database, a health information exchange (HIE) server, a storage that stores image files in the format of Digital Imaging and Communications in Medicine (DICOM), a picture archiving and communication system (PACS), a laboratory information system (LIS) including genomic data, a radiology information system (RIS), an antibiogram database, and/or a hospital guideline database. The medical data can include data relevant for prescription of an antibiotics and/or a diagnostic test such as the patients' medical history, the patients' most recent diagnoses, results of various measurements (e.g., body temperatures, blood pressures, etc.), and results of various laboratory tests (e.g., bacterial testing, culturing bacteria, antibiotic sensitivity testing, gram stain, etc.). The information may also include, for example, antibiogram information, guidelines for prescription and administering of the antibiotics, etc.

In some examples, the treating team sub-system can retrieve and aggregate the medical data based on receiving a trigger, such as a selection from the treating team to review the medical history of the patient.

In step 604, treating team sub-system 304 can generate a recommendation for at least one of a prescription of a first antibiotic of the plurality of antibiotics or an order of a first diagnostic test for a first patient of the plurality of patients, the recommendation being based on the medical data for the first patient, the antibiogram information, and the plurality of guidelines.

For example, treating team sub-system 304 (e.g., recommendation module 370) can generate a recommendation for a clinical decision of prescribing certain antibiotics and/or ordering certain diagnostic tests. The recommendation may include, for example, prescriptions of certain antibiotics, method of administering the antibiotics, dose, and/or duration of the therapy. The generation of the recommendation can be based on receiving a trigger. The trigger may be based on the same trigger as the trigger that causes treating team sub-system 304 (e.g., treating team data access interface 360a) to retrieve and aggregate the data from databases 328, or can be based on a different trigger based on, for example, the patient's medical data being displayed in treating team data access interface 360a, a command from the treating team member, an indication from databases 328 that certain new medical data of the patient (e.g., lab test results) are available, etc.

For example, in response to treating team data access interface 360a displaying the medical data of a patient, recommendation module 370 may transmit a query to databases 328 for updates (if any) of the medical data of the patient. If recommendation module 370 detects that certain new medical data relevant for a clinical decision of prescribing certain antibiotics and/or ordering certain diagnostic tests (e.g., lab test results) becomes available, recommendation module 370 can process the updated medical data of the patient and determine the recommendation for a clinical decision.

The recommendation can be generated based on various techniques, such as based on applying one or more rules on the medical data. The rules may indicate, for example, selecting one or more antibiotics that are effective to treat a patient's disease while minimizing pathogen resistance to the selected antibiotics. The rules can be applied to the patient's medical data, guidelines, and antibiogram information to generate a recommendation. The medical data can include, for example, clinical response, diagnostic test results (e.g. cultures and susceptibility testing), and other lab parameters (e.g., creatinine, drug levels).

As an example, referring back to FIG. 3B, based on a disease of the patient as indicated in the medical data of the patient, rules module 340a can retrieve a guideline graph from guidelines database 328b. Moreover, rules module 340a can traverse the guideline graph based on a state of the disease of the patient (e.g., whether or not the patient is admitted to the hospital, a degree of severity, presence/absence of comorbidities, etc.) and identify a list of candidate antibiotics that can be prescribed to the patient as a treatment for the disease, as well as the dosages of the candidate antibiotics, at the end of the traversal.

In addition, referring to FIG. 3C, rules module 370a can retrieve an antibiogram table based on a treatment location of the patient, and identify sections of the antibiogram table corresponding to the list of recommended antibiotics. In each section, rules module 370a can determine the degree of resistance of one or more pathogens that cause the patient's disease (as indicated in, for example, the lab test results of the patient) to the prescribed antibiotics. Rule module 370a can rank the recommended antibiotics based on the degrees of resistance, and include a subset of the list of recommend antibiotics having the lowest degrees of resistance, or having degrees of resistance below a threshold, in the recommendation.

In some examples, an alternate regiment ranking can be performed, which includes computing benefit score and a risk score for each alternative antibiotic therapy regiment, which can include one or more different antibiotics, ranking the alternate regiments based on a ratio between the benefit score and the risk score for each regiment, and selecting the regiment having the highest ratio as the intervention recommendation. The benefit score can be based on, for example, a susceptibility of the pathogen to the antibiotics, whereas the risk score can be based on, for example, whether any of the antibiotics in the regiment is redundant, a risk of the pathogen becoming resistant to a particular antibiotic in the regiment, etc. The risk score can also be computed based on a medical history of the patient (e.g., whether the patient has experienced resistance), suspected diagnosis, etc. In some examples, the recommendation can be generated based on performing a prediction operation. The goal of the prediction operation can include, for example, predicting when an antibiotic treatment is needed. The prediction can be based on determining the likelihood that a bacterial infection requires an antibiotic treatment versus the likelihood that the infection is a self-resolving infection, or that the disease of a patient is a non-infectious process. In some examples, the prediction can be based on a trade-off between “number needed to treat” (how many patients need to be treated with an antibiotic in order to benefit one patient?) versus “number needed to harm” (how many patients could be treated with an antibiotic before one experiences a treatment harm?).

In step 606, treating team sub-system 304 can provide, via an interface (e.g., treating team interface 360), access to the medical data of the first patient, a subset of the antibiogram information relevant to a medical condition of the first patient, and the recommendation, to facilitate a first clinical decision by the first member, the first clinical decision including at least one of prescribing a first dosage of the first antibiotic or ordering the first diagnostic test to the first patient.

Specifically, treating team interface 360 can be a mobile interface to be provided on a mobile device (e.g., smart phone, tablet, etc.), and can include a treating team data access interface 360a, which can select a subset of the medical data, and display the selected data in the data access interface, as shown in FIG. 5A. In some examples, treating team sub-system 304 (e.g., treating team treating team data access module 362, treating team data access interface 360a, etc.) can automatically perform a filtering operation based on a degree of relevancy of the data to a particular patient. For example, the treating team interface can provide antibiogram information based on a location where the patients are to be administered antibiotics (e.g., ICU). As another example, the treating team sub-system can automatically select a subset of patients based on the antibiotic profile, laboratory test results of the patients, medical history of the patients, intervention tracking status, etc., and provide access to the medical data of the subset of patients via the treating team data access interface. In some examples, the filtering operation can also be performed based on inputs from the user.

In addition to displaying the relevant data for a clinical decision about a first dose or an empiric therapy, the treating team sub-system can also generate, as part of CDS, a recommendation for the first dose or the empiric therapy. The treating team sub-system can generate the recommendation based on various types of medical data, such as a medical history of the patient including allergies to drugs, a suspected diagnosis of the patient, suspected pathogens causing a disease of the patient, a risk of drug resistance of the patient, laboratory test results of the patient, antibiogram information, guidelines, formulary restrictions and inventory status etc. The recommendation can be generated based on a trigger such as, for example, a command/request from a treating team member, an indication (e.g., a network message) that new medical data (e.g., laboratory test results) of the patient is available, etc. The treating team sub-system can display the recommendation in the treating team data access interface, to enable the user to have access to the basis of the recommendation. The treating team can then make a clinical decision (e.g., a prescription order) based on accepting or rejecting the recommendation. The recommendation can be displayed with the medical data to provide a basis for the recommendation, as shown in FIG. 5B.

As an example, referring back to FIG. 3B, based on a disease of the patient as indicated in the medical data of the patient, rules module 340a can retrieve a guideline graph from guidelines database 328b. Moreover, rules module 340a can traverse the guideline graph based on a state of the disease of the patient (e.g., whether or not the patient is admitted to the hospital, a degree of severity, presence/absence of comorbidities, etc.) and identify a list of candidate antibiotics that can be prescribed to the patient as a treatment for the disease, as well as the dosages of the candidate antibiotics, at the end of the traversal.

In addition, referring back to FIG. 3C, rules module 370a can retrieve an antibiogram table based on a treatment location of the patient, and identify sections of the antibiogram table corresponding to the list of recommended antibiotics. In each section, rules module 370a can determine the degree of resistance of one or more pathogens that cause the patient's disease (as indicated in, for example, the lab test results of the patient) to the prescribed antibiotics. Rule module 370a can rank the recommended antibiotics based on the degrees of resistance, and include a subset of the list of recommend antibiotics having the lowest degrees of resistance, or having degrees of resistance below a threshold, in the recommendation.

In addition, treating team sub-system 304 can output a clinical decision of prescribing certain antibiotics and/or ordering certain diagnostic tests. Treating team sub-system 304 can accept an input from a treating team member, via treating team interface 360, and generate the clinical decision based on the input. The input can be based on, for example, the recommendation generated in step 604. Treating team sub-system 304 can then send the clinical decision as a notification (e.g., a text message, a snoozing notification, etc.) to other treating team sub-systems 304 via treating team communication module 368.

In some examples, method 600 may further include steps 608 and 610.

In step 608, treating team sub-system 304 can receive, from an ASP team sub-system 302, an intervention recommendation to intervene in the first clinical decision. The intervention recommendation can be in the form of a notification, a text message, etc.

In step 610, treating team sub-system 304 can display the intervention recommendation. In some examples, the intervention recommendation can include a notification that can be displayed or output as other sensory output (e.g., a vibration, a tone, etc.) while the mobile device is in an idle state, as shown in FIG. 5C. Upon receiving a selection of the notification, treating team sub-system 304 can activate treating team interface 360 to display both the notification as well as medical data of the first patients. In some examples, the intervention recommendation can be a text message displayed in a text-messaging interface, as shown in FIG. 5D.

B. Example Method to be Performed by an ASP Team Sub-System

In addition, FIG. 6B illustrates an example of a method 650 which can be implemented by ASP team sub-system 302.

In step 652, ASP team sub-system 302 can retrieve, from a plurality of databases, medical data of a plurality of patients after being administered one or more antibiotics, antibiogram information that indicate resistance of pathogens to a plurality of antibiotics including the one or more antibiotics, and a plurality of guidelines related to treatment of certain diseases using the plurality of antibiotics.

Specifically, ASP data access module 316 of ASP team sub-system 302 can retrieve the medical data from one or more databases 328, such as an electronic medical record (EMR) database, a master patient index (MPI) services database, a health information exchange (HIE) server, a storage that stores image files in the format of Digital Imaging and Communications in Medicine (DICOM), a picture archiving and communication system (PACS), a laboratory information system (LIS) including genomic data, a radiology information system (RIS), an antibiogram database, and/or a hospital guideline database. ASP data access module 316 can source various types of data including, for example, various data of patients that are relevant for an intervention recommendation, such as their medical history, their most recent diagnoses, results of various measurements (e.g., body temperatures, blood pressures, etc.), and results of various laboratory tests (e.g., bacterial testing, culturing bacteria, antibiotic sensitivity testing, gram stain, etc.). The information may also include, for example, antibiogram information, guidelines for prescription and administering of the antibiotics, etc.

The ASP team sub-system can receive a trigger to retrieve and aggregate patients data for ASP intervention determination. The trigger can be based on, for example, a command from the ASP team to access the medical data of certain patients, a timer that indicates a review of patients' antibiotics usage/prescription by the ASP team is due, new antibiotics prescriptions have been entered for certain patients and the prescriptions have not been reviewed, etc.

In step 654, ASP team sub-system 302 can determine a triage ranking for each of the plurality of patients based on the medical data and the antibiogram information.

Specifically, to facilitate efficient review and intervention of the patients' therapies/tests, patient triage module 318 of ASP team sub-system 302 can determine a triage score for each patient, rank the patients based on their triage scores, and display a ranked list of patients in the ASP team interface. The triage score can indicate the urgency for reviewing the patient's treatment/test. The ASP team can then refer to the ranking to select a patient for review. The triage score can be a weighted average of various factors (e.g., availability of the most recent lab test result, lab/drug mismatch, restricted drug prescription, etc.), to indicate the urgency for reviewing the patient's antibiotics therapy. The weights can be determined by the ASP team and/or automatically by the ASP team sub-system based on, for example, prior interventions.

The triage score of a patient can be computed based on various techniques. Specifically, referring to FIG. 3F, a patient may be associated with a set of attributes 330 that can be used to determine the urgency for reviewing the patient's antibiotics/diagnostic test prescription. Each attribute can also be associated with a score s0, s1, etc. In some examples, the score of an attribute can be 0 for absence of the attribute and 1 for presence of the attribute. Other score values can also be assigned. In addition, each attribute can also be associated with a weight, such as w0, w1, w2, etc. A triage score can be computed based on a weighted average of the score, with a higher score meaning it is more urgent to review the patient's antibiotics/diagnostic test prescription and vice versa. The weights of the attributes can be determined in various ways. In some examples, the weights can be determined by the ASP team. For example, the weights can be assigned such that patients for whom new laboratory results are available, patients that warrant an active review as they now exhibit some characteristic indicating an opportunity to optimize the antibiotics they are being given (e.g., based on positive blood culture result, bug-drug mismatch showing that the current therapy is ineffective, etc.), and patients that are being administered multiple antibiotics, broad-spectrum drug, restricted antibiotics, or antibiotics on the shortage list, can receive review by the ASP team ahead of other patients who do not have these features. In some examples, the weights of the attributes can also be determined automatically by patient triage module 318 based on other criteria, such as a history of interventions. For example, if the ASP team has intervened the prescription of antibiotics/diagnostics tests for a patient having certain attributes before, patient triage module 318 can automatically increase the weights of those attributes.

In addition, the triage ranking of the patients can be based on other criteria. For example, as described above, ASP sub-system 302 (e.g., ASP notification module 322) can generate a notification if certain attributes of the patient, based on the latest laboratory test results and/or prescriptions, indicate that the patient warrants review from the ASP team. A number of notifications generated for each patient since the last time the patient's prescription is reviewed via ASP team sub-system 302 can be counted, and the patients can be ranked based on the number of notifications. The patient with the highest number of notifications can be ranked highest to indicate highest priority/urgency for reviewing the patient's prescription.

As another example, referring to FIG. 3F, the patients can be ranked based on the prescription of antibiotics. For example, for each patient, a composite antibiotics score that reflects the characteristics of the antibiotics currently prescribed to the patient can be computed, and patients having the same number of notifications can be further ranked based on the composite antibiotics score. The ranking based on composite antibiotics scores can be a secondary ranking after the number of notifications.

In some examples, referring to FIG. 3E, an antibiotic can be assigned a score that reflects whether it is a broad spectrum antibiotic that treats a broad range of pathogens, whether it is in shortage, whether it is restricted, etc. A restricted antibiotic can be assigned the highest score, which indicates the highest priority for review of its prescription. Each antibiotic prescribed to a patient can then be assigned a score, and the scores for a patient can be summed to generate an antibiotic composite score.

Referring to FIG. 3F, patients can be ranked based on the number of notifications, with patients having the highest number of notifications being ranked the highest (e.g., patients A and B). Among patients (e.g., patients A and B) having the same number of notifications, the patients can be further ranked based on their composite antibiotics scores, with patients having the highest composite antibiotic score ranked the highest. In some examples, the ranking can also be based on a duration in which the patients are administered a particular antibiotic. A patient who has been administered an antibiotic for a large number of consecutive days can be ranked higher than another patient who has been administered the antibiotic (or other antibiotics) for a fewer number of consecutive days.

In step 656, ASP sub-system 302 can display, via an interface accessible by a first member of an ASP team, a ranked patient list representing the plurality of patients and including at least a part of the medical data of the plurality of patients, the patient list being ranked based on the triage rankings of the plurality of patients, to facilitate an intervention recommendation by the first member of the ASP team to intervene a prescription of a first antibiotic to a first patient of the plurality of patients.

Specifically, the ranked patients list, as well as at least part of the medical data aggregated in step 652, can be displayed in data access interface 312a of ASP team interface 312, which can be a desktop interface, a mobile interface, etc. An example of the ranked patients list is shown in FIG. 4B. Upon displaying the ranked patients list (or receiving an instruction to display the ranked patients list), ASP data access module 316 can retrieve the medical data of the patients from databases 328. Moreover, upon receiving a selection of one of the patients in the ranked patients list, ASP data access module 316 can also retrieve additional data of the selected patient for display in data access interface 312a to facilitate an intervention commendation by the first member.

In some examples, ASP team sub-system 302 (e.g., ASP data access module 316, ASP data access interface 312a, etc.) can automatically perform a filtering operation based on a degree of relevancy of the data to the patient. For example, the ASP team sub-system can automatically select, for the patient, a subset of antibiogram information based on a location where the patient is to be administered antibiotics (e.g., ICU), laboratory test results of the patient (which can indicate which pathogen is causing an infectious disease of the patient), inventory of antibiotics, etc., to narrow down to a particular section of the antibiogram information for a particular pathogen against a narrow set of antibiotics for a particular setting, and provide the subset of antibiogram information for display in ASP data access interface 312a.

In some examples, ASP team sub-system 302 can generate, as part of a clinical decision support (CDS), a recommendation for intervention to facilitate the clinical decision. ASP team sub-system 302 can generate the recommendation based on, for example, the medical data of the selected patient, antibiogram information, guidelines, etc. ASP team sub-system 302 can display the recommendation in the ASP team data access interface concurrently with the medical data of the patient, to enable the user to have access to the basis of the recommendation, as shown in FIG. 4C.

ASP team sub-system 302 can generate various recommendations in various ways. Specifically, the intervention recommendation may include, for example, dosing changes, escalating/deescalating antimicrobials, conversion from IV to PO, etc. In some examples, the recommendation can be based on receiving an intervention recommendation from the ASP team via ASP team interface 312 (e.g., based on viewing a recommendation output by ASP team sub-system 302), or based on receiving a selection of a recommendation output by ASP team sub-system 302 to automatically generate an intervention recommendation.

The ASP team sub-system can generate the recommendation based on, for example, the medical data of the selected patient, antibiogram information, guidelines, etc., in response to a trigger. In some examples, the trigger may be the same trigger that causes the ASP team sub-system to retrieve and aggregate patients data for ASP intervention determination. In some examples, the trigger can be a different trigger based on, for example, an indication from the databases that new medical data of a patient being reviewed (e.g., lab test results, or other new medical data that have not been processed by the sub-system) are available. The indication can be in the form of a network message transmitted by the databases in response to a query transmitted by the ASP team sub-system.

In some examples, a rules module (e.g., rules module 340a) of the ASP team sub-system can generate a recommendation based on applying one or more rules. The rules may indicate, for example, selecting one or more antibiotics that are effective to treat a patient's disease while minimizing pathogen resistance to the selected antibiotics. The rules can be applied to the patient's medical data, guidelines, and antibiogram information to generate a recommendation.

As an example, referring back to FIG. 3B, based on a disease of the patient as indicated in the medical data of the patient, rules module 340a can retrieve a guideline graph from guidelines database 328b. Moreover, rules module 340a can traverse the guideline graph based on a state of the disease of the patient (e.g., whether or not the patient is admitted to the hospital, a degree of severity, presence/absence of comorbidities, etc.) and identify a list of candidate antibiotics that can be prescribed to the patient as a treatment for the disease, as well as the dosages of the candidate antibiotics, at the end of the traversal. Rules module 340a can determine whether the antibiotics prescribed to the patient are included in the list of recommended antibiotics, and whether the dosages of the prescribed antibiotics match the recommended dosages. If the prescribed antibiotics are not in the list of recommended antibiotics, or that the prescribed dosages do not match the recommended dosage, rules module 340a may determine a recommendation for intervening the antibiotics prescription (e.g., changing to a different antibiotic, changing the dosage, etc.).

In addition, referring to FIG. 3C, rules module 340a can retrieve an antibiogram table based on a treatment location of the patient, and identify sections of the antibiogram table corresponding to the antibiotics being prescribed. In each section, rules module 340a can determine the degree of resistance of one or more pathogens that cause the patient's disease (as indicated in, for example, the lab test results of the patient) to the prescribed antibiotics. If the degree of resistance exceeds a particular threshold, rules module 340a may also determine a recommendation for intervening the antibiotics prescription.

In another example, an alternate regiment ranking module (e.g., alternate regiment ranking module 340b) of ASP team sub-system 302 can compute a benefit score and a risk score for each alternative antibiotic therapy regiment, which can include one or more different antibiotics; rank the alternate regiments based on a ratio between the benefit score and the risk score for each regiment, and select the regiment having the highest ratio as the intervention recommendation. The benefit score can be based on, for example, a susceptibility of the pathogen to the antibiotics, whereas the risk score can be based on, for example, whether any of the antibiotics in the regiment is redundant, a risk of the pathogen becoming resistant to a particular antibiotic in the regiment, etc. The risk score can also be computed based on medical history of the patient (e.g., whether the patient has experienced resistance), suspected diagnosis, etc.

In another example, a prediction module (e.g., prediction module 340c) of ASP team sub-system 302 can generate a recommendation based on performing a prediction operation. The goal of the prediction operation can be to predict the likelihood/probability that a patient is on an inappropriate antibiotic therapy, or that intervention is needed. The prediction can be based on the antibiotic treatment received by other patients. For example, if a patient receives a certain antibiotic for five days, whereas other patients having the same infectious disease receive the same antibiotic for only two days and recover, recommendation module 340 may predict that the patient receives an inappropriate antibiotic therapy that has exceeded an expected duration (two days).

In some examples, method 650 further includes step 658, in which ASP team sub-system 302 can transmit an intervention recommendation based on the first clinical decision to a second system (e.g., treating team sub-system 304). The intervention recommendation can be in the form of a notification, a text message, etc. In some examples, ASP team sub-system 302 can generate a notification/text message of the intervention recommendation, which can be generated automatically based on the recommendation, or based on an input from the ASP team, and transmit the notification to a treating team sub-system (e.g., treating team sub-system 304) via real-time communication, such as text-messaging, voice call, etc. In some examples, the notification can be a snoozing notification so the treating team can act on the notifications at a later time. ASP team sub-system 302 can also track (automatically and/or based on inputs from the ASP team) the status of an intervention recommendation (e.g., whether a therapy change has been implemented, a diagnostic test has been ordered, etc.). In some examples, the ASP team communication interface can be in the form of a text-messaging interface, in which the notification, as well as a response to the notification from the treating team sub-system, can be displayed in the form of text messages. Moreover, ASP team sub-system 302 can display ASP team data access interface 312a and the ASP team communication interface 312b concurrently, which allows the users to communicate via text messages or voice while having access to the medical data, to facilitate the collaboration experience. All the information needed to act on the notification is also provided in a treating team interface (e.g., treating team interface 360) that displays/outputs the notification, as described in FIG. 6A and shown in FIG. 5B.

### V. Computer System

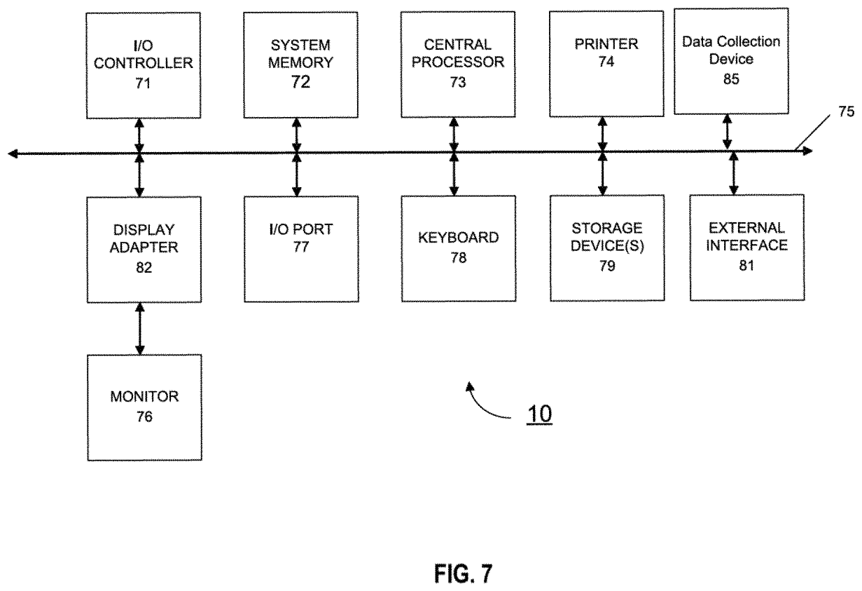

Any of the computer systems mentioned herein may utilize any suitable number of subsystems. Examples of such subsystems are shown in FIG. 7 in the computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 7 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire). For example, I/O port 77 or external interface 81 (e.g. Ethernet, Wi-Fi, etc.) can be used to connect the computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at the same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a,” “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

What is claimed is:

1. A method to support an antimicrobial stewardship program (ASP) within a heahthcare setting, the method being implemented by one or more computer processors and comprising:
retrieving, from a plurality of databases, medical data of a plurality of patients, antibiogram information that indicate resistance of pathogens to a plurality of antibiotics within a healthcare setting, and a plurality of guidelines related to treatment of certain diseases using the plurality of antibiotics;
generating a recommendation for at least one of a prescription of a first antibiotic of the plurality of antibiotics or an order of a first diagnostic test for a first patient of the plurality of patients, the recommendation being generated based on applying one or more rules to the plurality of guidelines, the antibiogram information, and the medical data for the first patient; and
providing, via an interface of a first system accessible by a first member of a treating team, access to the medical data of the first patient, a subset of the antibiogram information relevant to a medical condition of the first patient, and the recommendation to facilitate a first clinical decision by the first member, the first clinical decision including at least one of prescribing a first dosage of the first antibiotic or ordering the first diagnostic test to the first patient.

2. The method of claim 1, further comprising:
receiving a first trigger to retrieve the medical data of the plurality of patients from the plurality of databases, the first trigger comprising a first command via the interface to access the medical data of the plurality of patients;
responsive to receiving the first trigger, transmitting a query including identifiers of the plurality of patients to the plurality of databases to retrieve the medical data of the plurality of patients;
receiving a second trigger to generate a recommendation, the second trigger comprising at least one of: receiving an indication that the medical data of the first patient are displayed in the interface, receiving a second command via the interface to generate the recommendation, or receiving a network message from the plurality of databases indicating that new medical data of first patient is available; and
generating the recommendation responsive to receiving the second trigger.

3. The method of claim 1, wherein the recommendation is generated by:
retrieving, from the plurality of databases and based on a disease of the first patient indicated in the medical data of the first patient, a graph representing a guideline of the plurality of guidelines;
traversing the graph based on a status of the disease of the first patient to obtain a list of candidate antibiotics;
retrieving an antibiogram table of the antibiogram information from the plurality of databases based on a location identifier of a location where the first patient is to receive a antibiotics treatment, the antibiogram table comprising multiple sections, each section being associated with an antibiotic and listing degrees of resistances of different pathogens to the associated antibiotic;
identifying sections of the antibiogram table associated with the list of candidate antibiotics;
determining, based on the identified sections of the antibiogram, degrees of resistances of the list of candidate antibiotics to one or more pathogens that cause the patient's disease, the one or more pathogens being indicated in the medical data of the first patient;
ranking the list of candidate antibiotics based on the degrees of resistances;
selecting a recommended antibiotic from the list of candidate antibiotics based on the ranking; and
generating the recommendation including the recommended antibiotic and the associated dosage.

4. The method of claim 1, wherein the interface comprises a communication interface to enable real-time or asynchronous communication between the first member and other members of the treating team or between the first member and members of another team;
wherein the interface further comprises a data access interface configured to display data comprising at least one of the medical data of the first patient, the antibiogram information, or one or more recommended antibiotics; and
wherein the communication interface and the data access interface are provided concurrently to the first member.

5. The method of claim 4, wherein the communication interface enables the first member to send at least one of a prescription of the first dosage of the first antibiotic or an order of the first diagnostic test to a second member of the treating team.

6. The method of claim 4, wherein the communication interface enables the first member to respond to an intervention recommendation from a second system operated by an ASP team to change at least one of a prescription of the first dosage of the first antibiotic or the order of the first diagnostic test to the first patient.

7. The method of claim 6, wherein the interface is a first interface; and
wherein the method further comprises:
receiving the intervention recommendation from the second system;
displaying, in a second interface, a notification including the intervention recommendation; and
in response to receiving an input from the first member to respond to the notification, displaying the first interface including the communication interface and the data access interface to the first member.

8. The method of claim 1, wherein the plurality of databases comprise at least one of:
an electronic medical record (EMR) database, a master patient index (MPI) services database, a health information exchange (HIE) server, a storage that stores image files in the format of Digital Imaging and Communications in Medicine (DICOM), a picture archiving and communication system (PACS), a laboratory information system (LIS) including genomic data, a radiology information system (RIS), an antibiogram database, or a hospital guideline database.

9. The method of claim 1, wherein the medical data include at least one of: a medical history, a body temperature, a blood pressure, or a lab result at different time points.

10. The method of claim 1, wherein providing access to the medical data of the first patient comprises:
selecting a subset of the medical data; and
displaying the subset of the medical data in the interface;
wherein the subset of the medical data is selected based on at least one of: an input by the first member of the treating team, the subset of the medical data containing new test result for the first patient that has not been retrieved, or the subset of the medical data including laboratory measurement results that are relevant to the first clinical decision.

11. The method of claim 10, wherein the interface is a first interface; and
wherein the method further comprises:
receiving an indication that the new test result for the first member is stored at the plurality of databases;
displaying, in a second interface, a notification including the indication; and
in response to receiving an input from the first member to respond to the notification, displaying the subset of the medical data via the first interface to the first member.

12. The method of claim 11, further comprising:
selecting, based on a state of disease indicated in the medical data for the first patient, a subset of the plurality of guidelines; and
providing, via the interface, access to the subset of the plurality of guidelines.

13. The method of claim 1, wherein the recommendation for the prescription of the first antibiotic further includes a recommendation of at least one of: a route of administration of the first antibiotic, or a duration of a treatment course in which the first dosage of the first antibiotic is to be administered.

14. The method of claim 1, wherein the recommendation is generated based on at least one of: a medical history of the first patient, a suspected diagnosis of the first patient, suspected pathogens causing a disease of the first patient, a risk of drug resistance of the first patient, or laboratory test results of the first patient.

15. The method of claim 1, wherein the recommendation is generated based on:
determining a benefit-over-risk score for each of a plurality of alternative antimicrobial regimes, the risk being determined based on a likelihood of developing resistance to an antibiotics;
ranking the plurality of alternative antimicrobial regimes based on the benefit-over-risk scores; and
providing the recommended empirical antimicrobial regime based on the ranking.

16. The method of claim 1, wherein the recommendation for the empirical antimicrobial regime further includes a recommendation of when the first dosage of the first antibiotic is to be administered; and
wherein the recommendation is generated based on a ratio based on a first number needed to treat and a second number needed to harm, the first number indicating how many patients need to be treated with the first antibiotic in order to benefit one patient, the second number indicating how many patients can be treated with the first antibiotic before one experiences a treatment harm.

17. The method of claim 1, wherein the recommendation is generated based on at least one of: an antibiotic inventory of a hospital or of a clinic that is treating the first patient, or on a list of restricted drugs.

18. The method of claim 1, wherein the recommendation is generated based on a treatment history of a second patient of the plurality of patients and based on a comparison of symptoms between the first patient and the second patient.

19. The method of claim 1, wherein the recommendation is generated based on one or more rules; and
wherein at least one or more rules or the plurality of guidelines are editable by administrators of a hospital or a clinic.

20. The method of claim 1 comprising:
determining a triage ranking for each of the plurality of patients based on the medical data and the antibiogram information; and
displaying, via an interface accessible by a first member of an ASP team, a ranked patient list representing the plurality of patients and including at least a part of the medical data of the plurality of patients, the patient list being ranked based on the triage rankings of the plurality of patients, to facilitate a first clinical decision by the first member of the ASP team to intervene a prescription of a first antibiotic to the first patient of the plurality of patients.
